(19) United States
(12) Patent Application Publication
(10) Pub. No.: US 2026/0069534 A1
(43) Pub. Date: Mar. 12, 2026

(54) ORAL DELIVERY OF ACTIVE COMPOUNDS FOR TREATING GLYCOGEN STORAGE DISEASES

(71) Applicant: LYOTROPIC DELIVERY SYSTEMS LTD., Jerusalem (IL)

(72) Inventors: Nissim GARTI, Ramat HaSharon (IL); Reham ABU-GHOUSH, Jerusalem (IL); Sharon GARTI-LEVI, Modi'in (IL); Rotem EDRI, Eilat (IL); Rawan MUSLEH, Jerusalem (IL)

(73) Assignee: LYOTROPIC DELIVERY SYSTEMS LTD., Jerusalem (IL)

(21) Appl. No.: 19/105,482

(22) PCT Filed: Aug. 23, 2023
(86) PCT No.: PCT/IL2023/050895

§ 371 Date: Feb. 21, 2025

Related U.S. Application Data
(60) Provisional application No. 63/400,542, filed on Aug. 24, 2022.

(51) Int. Cl. A61K 9/00; A61K 31/4196; A61K 31/4436

(52) U.S. Cl. CPC A61K 9/006; A61K 31/4196; A61K 31/4436

(57) ABSTRACT

The present disclosure concerns formulations for oral delivery of at least one active agent of a compound according to formula (I) and/or (II) for the treatment of glycogen storage diseases, neurodegenerative disorders and autophagy-related conditions. The formulations are nano-structured formulations for increasing the bioavailability of the active agent after administration.

### TECHNOLOGICAL FIELD

The present disclosure concerns formulations for oral delivery of at least one active agent for the treatment of glycogen storage diseases, neurodegenerative disorders and/or autophagy-related conditions.

### BACKGROUND ART

References considered to be relevant as background to the presently disclosed subject matter are listed below:
PCT patent application publication no. WO2018/154578
PCT patent application serial no. PCT/IL2022/050187
Kapetanovic et al., “Effects of oral dosing paradigms (gavage versus diet) on pharmacokinetics and pharmacodynamics”, 2006, Chem Biol Interact, 164, 68-75
Kleyweg et al., “Interobserver Agreement In The Assessment Of Muscle Strength And Functional Abilities In Guillain-Barre Syndrome”, 1991, Muscle & Nerve, 14, 1103-1109

Acknowledgement of the above references herein is not to be inferred as meaning that these are in any way relevant to the patentability of the presently disclosed subject matter.

### BACKGROUND

Glycogen is a branched polysaccharide, composed of glucose units linked by α1-4 glycoside bonds to form the linear chains which hare further connected by α1-6 glycoside bonds to form the branching junctions.

Glycogen is primarily stored in the liver and muscles but can also be found in lower levels in the kidney, heart, and brain. In the liver, it provides an energy source under fasting, and in the muscles glycogen serves as an immediate reserve source of available glucose.

Glycogen synthesis begins with self-glycosylation of an oligosaccharide primer of glycogenin. Glycogen elongation involves glycogen-synthase that catalyzes the formation of α1-4 glycoside bonds and glycogen branching enzyme (GBE) that catalyzes the formation of α1-6 glycoside bonds and glycogen. The degradation of glycogen occurs through two different pathways, one in the cytosol (referred to as “glycogenolysis”) and another in the lysosome, called “glycogen autophagy” or “glycophagy”. The breakdown of glycogen via glycogenolysis involves glycogen phosphorylase (GP) and glycogen debranching enzyme (GDE), while the mechanism of glycophagy is mediated by acid alpha glycosidase (GAA).

The importance of glycogen metabolism is highlighted not only in context of energy maintenance, but also as it is associated with several congenital disorders caused by abnormal functioning of the enzymes that control the synthesis, degradation, and regulation of glycogen. These disorders, which in most cases are inherited in an autosomal recessive fashion, are collectively termed Glycogen Storage Disorders (GSDs). Today there are 16 known subtypes in the medical community of GSDs, including Lafora and Danon diseases, all appear as various clinical presentations.

Recently, several compounds have been designed for treating GSDs and described in PCT patent publication WO2018/154578. These compounds have shown promising activity in treating GSDs.

### GENERAL DESCRIPTION

The present disclosure provides formulations for oral delivery of active compounds for treating GSDs, as well as treating neurodegenerative disorders or conditions associated with lysosomal storage or autophagy-misregulation. The formulations of this disclosure are designed to enable increased loading of the active compounds, while maintaining high tolerability and improved bioavailability.

The formulations of this disclosure are formulated to stabilize the active compound in a water-less nanostructured formulation, while permitting full dilutability within aqueous liquids, such that once orally administered, the formulations homogenously disperse in the aqueous phase (e.g. stomach fluids), to form dispersed nanostructures in which the active compound is captured and stabilized. Such capturing permits stabilization of the active compound in the formulation, and once administered, release of the active compound from the nanostructures to ensure delivery of high effective doses of the active compound over time. The inventors have surprisingly found that by utilizing a combination of solvent(s), co-surfactant(s) and hydrophilic surfactant(s) which form a substantially hydrophilic delivery system, enables formation of nanostructures in water-less formulation, as well as in-situ formation of nanostructures after administration while stabilizing high loads of the lipophilic active compounds disclosed herein.

By one of its aspects, the present disclosure provides a pharmaceutical formulation for oral delivery of a compound of formula (I) or a pharmaceutically acceptable salt, isomer or tautomer thereof,

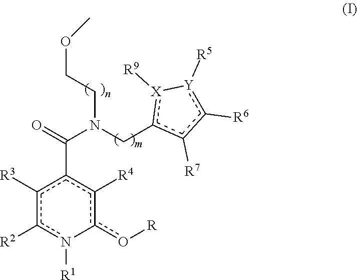

[symbol] represents a single or a double bond,
n and m are integers, each being independently 1, 2 or 3;
R and R¹ are each independently hydrogen or is absent;
R², R³, R⁴, R⁵, R⁶, R⁷, R⁸ and R⁹, are each hydrogen, or are each being independently selected from alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, and sulfonamide, each being further substituted or non-substituted; and one of X and Y is S, while the other of X and Y is C; provided that when X is S then R⁹ is absent, and when Y is S then R⁵ is absent;
   wherein the formulation comprises:
   a) said compound of formula (I) or a pharmaceutically acceptable salt, isomer or tautomer thereof;
   b) at least one hydrophilic surfactant in a total amount ranging between about 10 wt % and 50 wt %;
   c) at least one solvent in a total amount of at least about 20 wt %;
   d) at least one co-surfactant; and
   e) at least one oil in an amount of between 0 wt % and about 5 wt % of the formulation;
   the weight ratio of said at least one solvent to said at least one hydrophilic surfactant ranges between about 1.25:1 and about 1:3.

In some embodiments, the compound of formula (I) is a compound of formula (I′) or a pharmaceutically acceptable salt, isomer or tautomer thereof,

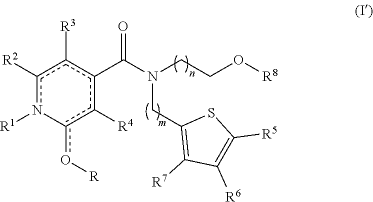

[symbol] represents a single or a double bond,
n and m are integers, each being independently 1, 2 or 3;
R and R¹ are each independently hydrogen or is absent; and
R², R³, R⁴, R⁵, R⁶, R⁷ and R⁸ are each hydrogen, or
R², R³, R⁴, R⁵, R⁶, R⁷ and R⁸ are each being independently selected from alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, and sulfonamide, each being further substituted or non-substituted.

The formulations of this disclosure are designed for oral delivery of the active compound, i.e. delivery of the active compound by swallowing, to obtain a systemic pharmacological effect. The formulations are typically in liquid form and can be administered as a liquid, a gel, a suspension, or encapsulated in a liquid gel or soft gel capsule.

The formulations of this disclosure, due to their unique formulatory composition, enable stably loading the formulation with the active compound in concentrations of at least 0.5 wt %, for example at least about 1 wt %, at least about 2 wt % wt %, at least about 3 wt %, at least about 4 wt %, or even at least about 5 wt % of the formulation.

By some embodiments, the formulation comprises up to about 10 wt % of said compound of formula (I) or a pharmaceutically acceptable salt, isomer or tautomer thereof.

The inventors have found that a combination of hydrophilic surfactant(s), co-solvent(s) and solvent(s) enables the high loading of the active compound into the formulation and stabilization thereof for prolonged period of time, while permitting the spontaneous formation of nanostructures both in the water-less formulation and when mixed with an aqueous liquid (e.g. stomach fluid after administration). In the formulations of this disclosure, the balance of ingredients permits high load and capturing of the lipophilic active agent in the predominantly hydrophilic formulation for prolonged time periods, permitting a long shelf life with minimal phase separation and/or sedimentation.

The formulations of this disclosure are typically in a concentrated form, typically water free concentrates, that are stably dilutable by an aqueous medium (namely without substantial increase in droplets size or phase separation when diluted). The concentrate form is stable for prolonged periods of time, which lacks a microorganisms' life-supporting environment, and is readily dilutable in aqueous media as will be further explained below.

Hence, the formulations disclosed herein are typically devoid of water. The formulations are designed to permit spontaneous formation of nanostructures, both in the concentrate form (i.e. without presence of water) and when mixed with an aqueous liquid (after administration or for the purpose of administration), as will be further detailed below.

In the formulations of this disclosure, the fine balance between the surfactants and co-surfactants/solvents imparts the formulations (being devoid of water) with physical stability in the presence of the pharmaceutically active compounds at high concentration, as such balance was found to promotes solubilization of the active compound. At the same time, the ratio between the surfactants and co-surfactants/solvents permits the dilution capacity of the formulation in aqueous liquids by allowing the formation of extremely small droplets of less than about 10 nm.

The term hydrophilic surfactant(s) refers to surface-active agents which have a hydrophilic head group and lipophilic tails that are capable of arranging into nanostructures in an aqueous medium. The inventors have found that a combination of hydrophilic surfactants with co-surfactants and solvents at specific ratio ranges and total concentrations are capable of spontaneously forming stable nanostructures which stabilize the active compound in the formulation in a water-less concentrate, as well as solubilize the active compound into nanostructures when mixed with an aqueous liquid.

By some embodiments, the at least one hydrophilic surfactant is selected from ethoxylated fatty acids, ethoxylated castor oil and hydrogenated derivatives thereof, polysorbates, ethoxylated alkyl ethers, ethoxylated monoglycerides, polyglycerol esters, sucrose esters, and combinations thereof.

According to some embodiments, the formulation comprises at least one first hydrophilic surfactant selected from ethoxylated castor oil and hydrogenated derivatives thereof (e.g. polyoxyl 35 castor oil, polyoxyl 40 hydrogenated castor oil, polyoxyl 60 castor oil, polyoxyl 60 hydrogenated castor oil), and at least one second hydrophilic surfactant selected from polysorbates (polysorbate 20, polysorbate 60, polysorbate 80), and ethoxylated monoglycerides (caprylocaproyl polyoxyl-8 glycerides, lauryl polyoxyl 32 glycerides, stearoyl poloxyl 32 glycerides, etc.).

As noted, at least one hydrophilic surfactant is present in the formulation in an amount of between about 10 wt % and about 50 wt %. By some embodiments, the at least one hydrophilic surfactant in a total amount ranging between about 20 wt % and 50 wt %. According to other embodiments, the at least one hydrophilic surfactant in a total amount ranging between about 25 wt % and 50 wt %.

The formulation comprises at least one co-surfactant. Co-surfactant should be understood to encompass any hydrophilic, lipophilic or amphiphilic agent, different from said hydrophilic surfactant(s), which contributes (together with the surfactants) to lowering of the interfacial tension between an oily phase and an aqueous phase to almost zero (or zero), allowing for the formation of thermodynamically stable nanostructures. Hence, the combination of surfactants and co-surfactants permits stabilization of the formulation both kinetically and thermodynamically.

By some embodiments, the at least one co-surfactant is selected from polyethylene glycol 200, polyethylene glycol 400, polyethylene glycol 600, propylene glycol, phospholipids (such as phosphatidylcholine), diethyleneglycol monoethyl ether, and combinations thereof.

By some embodiments, the at least one co-surfactant is present in the formulation in total amount ranging between about 8 wt % and about 45 wt %. According to some other embodiments, the at least one co-surfactant is present in the formulation in a total amount ranging from about 8 wt % to about 30 wt %, or even between about 8 wt % and 25 wt %.

By some embodiments, the weight ratio between the total hydrophilic surfactants and the total co-surfactants in the formulation ranges between about 3:1 and about 1:3. By some embodiments, the weight ratio between the total hydrophilic surfactants and the total co-surfactants in the formulation is about 3:1, 2.9:1, 2.8:1, 2.7:1, 2.6:1, 2.5:1, 2.4:1, 2.3:1, 2.2:1, 2.1:, 2.0:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, or about 1:3.

By some embodiments, the weight ratio between the total hydrophilic surfactants and the total co-surfactants in the formulation ranges between about 2.5:1 and about 1:1.5. By yet further embodiments, the weight ratio between the total hydrophilic surfactants and the total co-surfactants in the formulation ranges between about 2:1 and about 1:1.

The formulation also comprises relatively large amounts, typically at least 20 wt %, of at least one solvent. The solvent is an organic solvent, typically polar, that is at least partially water miscible and is suitable for assisting the solubilization of the active compound in the formulation, as well as into the nanostructure.

By some embodiments, the formulation comprises said at least one solvent in a concentration ranging between about 20 wt % and about 45 wt %. By some embodiments, the formulation comprises said at least one solvent in a concentration of between about 20 wt % and 35 wt %.

According to some embodiments, said at least one solvent is selected from ethanol, methanol, n-propanol, benzyl alcohol, and combinations thereof.

By some embodiments, the total amount of solvents and co-surfactants in the formulation is at least about 45 wt %. According to some embodiments, the total amount of solvents and co-surfactants in the formulation is at least about 50 wt %. According to other embodiments, the total amount of solvents and co-surfactants in the formulation is at least about 52 wt %. By some other embodiments, the total amount of solvents and co-surfactants in the formulation is at least 55 wt %.

By some embodiments, the weight ratio between the total solvents and the total co-surfactants ranges between about 3:1 and 1:2. By some embodiments, the weight ratio of the total amount of solvents to the total amount of co-surfactants ranges between about 2:1 and about 1:1.5.

By some embodiments, the weight ratio of the total amount of solvents to the total amount of co-surfactants is about 3:1, 2.8:1, 2.6:1, 2.4:1, 2.2:1, 2:1, 1.8:1, 1.6:1, 1.4:1, 1.2:1, 1:1, 1:1.2, 1:1.4, 1:1.6, 1:1.8, or about 1:2.

As noted, the weight ratio of the total amount of solvents to the total amount of hydrophilic surfactants ranges between about 1.25:1 and about 1:3. By some embodiments, the weight ratio of the total amount of solvents to the total amount of hydrophilic surfactants ranges between about 1:1 and about 1:2.

By some embodiments, the weight ratio of the total amount of solvents to the total amount of hydrophilic surfactants is about 1.25:1, 1.2:1, 1.15:1, 1.1:1, 1.05:1, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, or about 1:3.

The formulations comprise between 0 wt % and about 5 wt % oil. By some embodiments, the at least one oil is present in the formulation in a concentration of no more than 4 wt %. According to some embodiments, the formulations are devoid of oil.

The term oil refers to a lipophilic agent which is immiscible in water and is capable of forming distinct domains when introduced into an aqueous liquid. In some embodiments, the at least one oil is selected from short chain triglycerides and medium chain triglycerides.

In some embodiments, the formulations may further comprise various additives approved for pharmaceutical uses, such as pH adjusting agents and buffers, neutralizing agents, emollients, humectants, preservatives, antioxidants, taste masking agents, taste modifying agents, sweeteners, flavor additives, and any other suitable non-active pharmaceutical additive.

The formulations of the present disclosure are designed as pharmaceutical formulations for oral delivery of a compound of formula (I) or a pharmaceutically acceptable salt, isomer or tautomer thereof.

According to some embodiments, n and m in formula (I) are 1.

By some embodiments, R², R⁷ and R⁸ in formula (I) are each methyl.

According to some embodiments, the compound of formula (I) is at least one compound of formula (IA) or (IB):

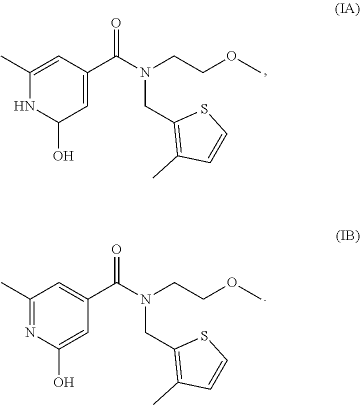

According to other embodiments, the formulation comprises two or more compounds of formula (I).

As used herein, alkyl, alkenyl alkynyl carbon chains, if not otherwise specified, contain from 1 to 20 carbons, and can be straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isohexyl, allyl (propenyl) and propargyl (propynyl).

C₁₋₆ alkyl should be understood to encompass any straight or branched alkyl moiety having 1, 2, 3, 4, 5 or 6 carbon atoms.

Cycloalkyl refers to a saturated mono-cyclic or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion.

Heterocyclyl refers to a monocyclic or multicyclic non-aromatic ring system, in one embodiment of 3 to 10 members, where one or more of the atoms in the ring system is a heteroatom, that is, an element other than carbon, including but not limited to, nitrogen, oxygen or sulfur.

Alkoxy refers to an —O-alkyl or an —O-cycloalkyl, as defined herein; tioalkoxy refers to an —S-alkyl or an —S-cycloalkyl, as defined herein.

Aryl refers to aromatic monocyclic or multicyclic carbon groups containing from 5 to 19 carbon atoms, namely having conjugated pi-electron system.

Heteroaryl refers to a monocyclic or multicyclic aromatic ring system, in certain embodiments, of about 5 to about 19 members where one or more of the atoms in the ring system is a heteroatom, that is, an element other than carbon, including but not limited to, nitrogen, oxygen or sulfur.

Aryloxy refers to an —O-aryl or an —O-heteroaryl, as defined herein; thioaryloxy refers to an —S-aryl or an —S-heteroaryl, as defined herein.

Hydroxy refers to an —OH group.

Thiohydroxy (or thiol) refers to an —SH group.

Amino refers to primary, secondary or tertiary amines (—NR′R″, R′ and R″ are independently hydrogen, alkyl, cycloalkyl, alkenyl, aryl, heteroaryl, or a heterocyclyl as defined herein), where the point of attachment is through the nitrogen atom which is substituted with C₁-C₆ straight or branched alkyl. In case of a tertiary amine, the substituents can be the same or different.

Nitro refers to an —NO₂ group.

Halo (or halogen or halide) refers to F, Cl, Br or I. Haloalkyl refers to an alkyl group in which one or more of the hydrogen atoms are replaced by halogen. Such groups include, but are not limited to, trihalomethyl.

Cyano refers to —C≡N group.

Amide refers to a refers to the divalent group —C(O)NH₂.

Carboxy refers to a —C(O)—OR′ group, wherein R′ is hydrogen, alkyl, cycloalkyl, alkenyl, aryl, heteroaryl, or a heterocyclyl as defined herein.

Sulfonyl refers to a —S(O)₂—R′ group, wherein R′ is hydrogen, alkyl, cycloalkyl, alkenyl, aryl, heteroaryl, or a heterocyclyl as defined herein.

Sulfinyl refers to a —S(O)—R′ group, wherein R′ is hydrogen, alkyl, cycloalkyl, alkenyl, aryl, heteroaryl, or a heterocyclyl as defined herein.

Sulfonamide refers to a —S(O)₂—NR′R″ group, R′ and R″ are independently hydrogen, alkyl, cycloalkyl, alkenyl, aryl, heteroaryl, or a heterocyclyl as defined herein.

The term pharmaceutically acceptable salt(s), as used herein, means those salts of compounds of this disclosure that are safe and effective for pharmaceutical use in mammals and that possess the desired biological activity. Pharmaceutically acceptable salts include salts of acidic or basic groups present in compounds of this disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e. 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

The compounds described herein comprise one or more chiral atoms, or may otherwise be capable of existing as isomers, e.g. two enantiomers or as two or more diastereomers. Accordingly, the compounds can include mixtures of isomers as well as purified isomers or enantiomerically enriched mixtures. Furthermore, the compounds can include mixtures of diastereomers, as well as purified stereoisomers or diastereomerically enriched mixtures. It is also noted that the compounds may form tautomers, isolated or in any mixture thereof.

According to another aspect of this disclosure, there is provided a pharmaceutical formulation for oral delivery of a compound of formula (II) or a pharmaceutically acceptable salt, isomer or tautomer thereof,

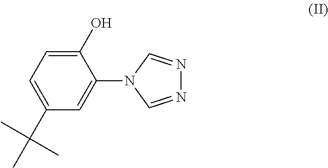 [SMILES: CC(C)(C)c1ccc(O)c(-n2cnnc2)c1]

wherein the formulation comprises:
   a) said compound of formula (II) or a pharmaceutically acceptable salt, isomer or tautomer thereof,
   b) at least one hydrophilic surfactant in a total amount ranging between about 10 wt % and 70 wt %,
   c) at least one solvent in a total amount of at least about 15 wt %;
   d) at least one co-surfactant, and
   e) at least one oil in an amount of between 0 wt % and about 5 wt % of the formulation,
   the weight ratio of said at least one solvent to said at least one hydrophilic surfactant ranges between about 1:1 and about 1:7.

By some embodiments, the formulation can comprise a mixture of at least one compound of formula (I) and a compound of formula (II).

By some embodiments, the formulation comprises up to about 10 wt % of said compound of formula (II) or a pharmaceutically acceptable salt, isomer or tautomer thereof.

By some embodiments, the at least one hydrophilic surfactant is selected from ethoxylated fatty acids, ethoxylated castor oil and hydrogenated derivatives thereof, polysorbates, ethoxylated alkyl ethers, ethoxylated monoglycerides, polyglycerol esters and sucrose esters, and combinations thereof. According to some embodiments, the formulation comprises at least one first hydrophilic surfactant selected from ethoxylated castor oil and hydrogenated derivatives thereof (e.g. polyoxyl 35 castor oil, polyoxyl 40 hydrogenated castor oil, polyoxyl 60 castor oil, polyoxyl 60 hydrogenated castor oil), and at least one second hydrophilic surfactant selected from polysorbates (polysorbate 20, polysorbate 60, polysorbate 80), and ethoxylated monoglycerides (caprylocaproyl polyoxyl-8 glycerides, lauryl polyoxyl 32 glycerides, stearoyl poloxyl 32 glycerides, etc.).

By some embodiments, the at least one hydrophilic surfactant is in a total amount ranging between about 20 wt % and 70 wt % of the formulation of compound (II). According to other embodiments, the at least one hydrophilic surfactant in a total amount ranging between about 30 wt % and 70 wt %. By some embodiments, the formulation of a compound of formula (II) comprises said at least one hydrophilic surfactant in a total amount ranging between about 20 wt % and 50 wt %.

By some embodiments, the at least one co-surfactant is selected from polyethylene glycol 200, polyethylene glycol 400, polyethylene glycol 600, propylene glycol, phosphatidylcholine, diethyleneglycol monoethyl ether, and combinations thereof.

By some embodiments, the at least one co-surfactant is present in the formulation of compound of formula (II) in total amount ranging between about 8 wt % and about 45 wt %. By other embodiments, the at least one co-surfactant is present in the formulation of compound of formula (II) in total amount ranging between about 8 wt % and about 35 wt %.

By some embodiments, the weight ratio between the total hydrophilic surfactants and the total co-surfactants in the formulation of a compound of formula (II) ranges between about 7:1 and about 1:3. By some embodiments, the weight ratio between the total hydrophilic surfactants and the total co-surfactants in the formulation ranges between about 7:1 and about 1:1, e.g. about 7:1, 6.5:1, 6:1, 5.5:1, 5:1, 4.5:1, 4:1, 3.5:1, 3:1, 2.5:1, 2:1, 1.5:1, or 1:1.

The formulation of compound of formula (II) also comprises relatively large amounts, typically at least 15 wt %, of at least one solvent. By some embodiments, the formulation of compound of formula (II) comprises said at least one solvent in a concentration ranging between about 15 wt % and about 45 wt %. According to some embodiments, the formulation of compound of formula (II) comprises said at least one solvent in a concentration ranging between about 15 wt % and about 35 wt %.

According to some embodiments, said at least one solvent is selected from ethanol, methanol, n-propanol, benzyl alcohol, and combinations thereof.

By other embodiments, the weight ratio of said at least one solvent to said at least one hydrophilic surfactant ranges between about 1.1:1 and about 1:5 in said formulation of a compound of formula (II). At some embodiments, said weight ratio of said at least one solvent to said at least one hydrophilic surfactant ranges between about 1.1:1 and about 1:3.

By some embodiments, the total amount of solvents and co-surfactants in the formulation of compound of formula (II) is at least about 25 wt %.

The formulations of compound of formula (II) comprise between 0 wt % and about 5 wt % oil, preferably up to 2 wt % of oil, and even more preferably are devoid of oil.

In some embodiments, the formulations of compounds of formula (I), formula (I′) and/or formula (II) may further comprise various additives approved for pharmaceutical uses, such as pH adjusting agents and buffers, neutralizing agents, emollients, humectants, preservatives, antioxidants, taste masking agents, taste modifying agents, sweeteners, flavor additives, and any other suitable non-active pharmaceutical additive.

The formulations of compounds of formula (I), formula (I′) and/or formula (II) of the present disclosure are designed as pharmaceutical formulations for oral delivery of a compound of formula (I), formula (I′) and/or formula (II) or a pharmaceutically acceptable salt, isomer or tautomer thereof.

As noted, the formulations of compounds of formula (I), formula (I′) and/or formula (II) of this disclosure are designed to be stable (thermodynamically and kinetically) for prolonged periods of time. The formulations of this disclosure form nanostructures both when in concentrate form (i.e. devoid of water) and once mixed with an aqueous liquid. The nanostructures stabilize and capture the active compound, to permit its containment within the formulation before diluting with an aqueous liquid, as well as release from the nanostructures after administration (i.e. after dilution).

The careful balance between the hydrophilic surfactant(s), co-surfactant(s) and solvent(s) permits spontaneous formation of the nanostructures, in which the active compound is solubilized and stabilized. The combination of hydrophilic surfactant(s), co-surfactant(s) and solvent(s) facilitates full coverage of the interface between the nanostructures and the aqueous diluent at high water dilutions of the formulation. In other words, the combination of hydrophilic surfactants, co-surfactants and solvents alters the effective critical packing parameter (ECPP) of the interface, facilitating the control of the hydrophilicity/hydrophobicity of the surfactants, depending on the amount of water, thus increasing stability of the nanostructures.

The formulations of this disclosure can be administered as-is, i.e. in concentrate form, readily dilutable in-situ after administration by stomach fluids. Alternatively, the formulation can be administered in a diluted form, by diluting the formulation with one or more aqueous diluents before administration.

Thus, by another one of its aspects, the disclosure provides a preparation for oral delivery of a compound of formula (I) or a pharmaceutically acceptable salt, isomer or tautomer thereof, the preparation comprises nanodroplets of a formulation comprising said compound of formula (I) or a pharmaceutically acceptable salt, isomer or tautomer thereof disclosed herein, dispersed in a continuous phase comprising at least one aqueous diluent.

A further aspect provides a preparation for oral delivery of a compound of formula (I′) or a pharmaceutically acceptable salt, isomer or tautomer thereof, the preparation comprises nanodroplets of a formulation comprising said compound of formula (I′) or a pharmaceutically acceptable salt, isomer or tautomer thereof disclosed herein, dispersed in a continuous phase comprising at least one aqueous diluent.

A further aspect provides a preparation for oral delivery of a compound of formula (II) or a pharmaceutically acceptable salt, isomer or tautomer thereof, the preparation comprises nanodroplets of a formulation comprising said compound of formula (II) or a pharmaceutically acceptable salt, isomer or tautomer thereof, dispersed in a continuous phase comprising at least one aqueous diluent.

It is generally noted that, unless specifically noted otherwise, the term formulation is used to denote a water-free composition (i.e. a concentrate form), while the term preparation means to denote a diluted form of the formulation.

The nanodroplets (or nanostructures) are droplets composed of the formulation that capture and stabilize the pharmaceutically active compound. The nanostructures are typically in the form of liquid droplets, having an average diameter of at most 50 nm (nanometers), in which the hydrophilic surfactants and co-surfactants form an interface between a continuous phase of the solvents (when in water-less concentrate form) or the continuous aqueous phase and the oil core. Without wishing to be bound by theory, the active compound is located at the interface, such that at least some of the active compound is physically captured between the heads of the co-surfactants to stabilize it within the nanostructures.

According to some embodiments, the nanodroplets have an average droplet size ranging between about 5 nm and 50 nm.

The term average size refers to the arithmetic mean of measured diameters of the droplets. Where the droplets are not spherical, the calculation of the average size is based on an equivalent sphere about the largest dimension of the particles.

By some embodiments, the nanodroplets are substantially mono-disperse. The formulations and preparations are typically transparent (or substantially transparent) due to their mono-dispersed submicronic nanostructures' size, maintaining their transparency for a prolonged period of time. This permits easy detection of changes in the formulation's and/or preparation's stability (as phase separation, bioactive precipitation, and/or coalescence of droplets will cause detectable clouding).

According to some embodiments, the at least one aqueous diluent is selected from water, water for injection, saline, dextrose solution, and a buffer solution.

By another aspect, there is provided a formulation or preparation as disclosed herein, for use in treating glycogen storage disease (GSD).

By a further aspect, there is provided a method of treating a glycogen storage disease (GSD), comprising administering an effective amount of a formulation or preparation as disclosed herein, to a patient in need thereof.

According to some embodiments, the GSD is associated with glycogen-branching enzyme deficiencies. The glycogen-branching enzyme deficiencies means a disease or disorder characterized by deposition, accumulation or aggregation of polyglucosan bodies in muscle, nerve and/or other tissues of the body.

According to some other embodiments, the GSD is GSD type 0, GSD type I, GSD type II, GSD type III, GSD type IV, GSD type V, GSD type VI, GSD type VII, GSD type VIII, GSD type IX, GSD type X, GSD type XI, GSD type XII, GSD type XIII, GSD type XIV, or GSD type XV.

According to other embodiments, the GSD is adult polyglucosan body disorder (APBD). Andersen disease, Forbes disease, or Danon disease.

By a further aspect of this disclosure, there is provided a formulation or preparation as disclosed herein, for use in treating a disease or condition associated with lysosomal storage.

By yet a further aspect, this disclosure provides a method of treating a disease or condition associated with lysosomal storage, comprising administering an effective amount of a formulation or preparation as disclosed herein, to a patient in need thereof. Lysosomal storage disorders (LSDs) are a group of inherited diseases characterized by lysosomal dysfunction and neurodegeneration. These disorders are typically caused by single gene defects, primarily in specific enzymes that are required for normal breakdown of glycosaminoglycans (GAGs). Such defects make the cell unable to excrete carbohydrate residues, causing accumulation of the residues in the lysosomes within the cell, thereby causing disruption of the normal function of the cell. Exemplary lysosomal storage disorders are Sphingolipidoses, Ceramidase (e.g. Farber disease, Krabbe disease), Galactosialidosis, gangliosidoses including Alpha-galactosidases (e.g. Fabry disease (alpha-galactosidase A), Schindler disease (alpha-galactosidase B)), Betagalactosidase (e.g. GM1 gangliosidosis, GM2 gangliosidosis, Sandhoff disease, Tay-Sachs disease), Glucocerebrosidoses (e.g. Gaucher disease (Type I, Type II, Type III), Sphingomyelinase (e.g. Lysosomal acid lipase deficiency, Niemann-Pick disease), Sulfatidosis (e.g. Metachromatic leukodystrophy, Multiple sulfatase deficiency), Mucopolysaccharidoses (e.g. Type I (MPS I (Hurler syndrome, Scheie syndrome, Hurler-Scheie syndrome), Type II (Hunter syndrome), Type III (Sanfilippo syndrome), Type IV (Morquio), Type VI (Maroteaux-Lamy syndrome), Type VII (Sly syndrome), Type IX (hyaluronidase deficiency)), mucolipidoses (e.g. Type I (sialidosis), Type II (I-cell disease), Type III (pseudo-Hurler polydystrophy/phosphotransferase deficiency), Type IV (mucolipidin 1 deficiency)), lipidoses (e.g. Niemann-Pick disease), Neuronal ceroid lipofuscinoses (e.g. Type 1 Santavuori-Haltia disease/infantile NCL (CLN1 PPT1)), Type 2 Jansky-Bielschowsky disease/late infantile NCL (CLN2/LINCL TPP1), Type 3 Batten-Spielmeyer-Vogt disease/juvenile NCL (CLN3), Type 4 Kufs disease/adult NCL (CLN4), Type 5 Finnish Variant/late infantile (CLN5), Type 6 Late infantile variant (CLN6), Type 7 CLN7, Type 8 Northern epilepsy (CLN8), Type 8 Turkish late infantile (CLN8), Type 9 German/Serbian late infantile, Type 10 Congenital cathepsin D deficiency (CTSD)), Wolman disease, Oligosaccharidoses (e.g. Alpha-mannosidosis, Beta-mannosidosis, Aspartylglucosaminuria, Fucosidosis), lysosomal transport diseases (e.g. Cystinosis, Pycnodysostosis, Salla disease/sialic acid storage disease, Infantile free sialic acid storage disease), Type II Pompe disease, Type lib Danon disease), Cholesteryl ester storage disease, and the like.

By some embodiments, the disease or condition associated with lysosomal storage is selected from Gaucher disease, Fabry disease, Tay-Sachs disease, Mucopolysaccharide (MPS) disorders, aspartylglucosaminuria, GMI-gangliosidosis, Krabbe (globoid cell leukodystrophy or galactosylceramide lipodosis), metachromatic leukodystrophy, Sandhoff disease, mucolipidosis type II (I-cell disease), mucolipidosis type IIIA (pseudo-Hurler polydystrophy), Niemann-Pick disease type C2 and C1, Danon disease, free sialic acid storage disorder, mucolipidosis type IV, multiple sulfatase deficiency (MSD), metabolic disorders, obesity, type II diabetes, and insulin resistance.

By yet another aspect, there is provided a formulation or preparation as disclosed herein, for use in treating a disease or condition associated with autophagy-misregulation.

By still a further aspect, there is provided a method of treating a disease or condition associated with autophagy-misregulation, comprising administering an effective amount of a formulation or preparation as disclosed herein, to a patient in need thereof.

Autophagy refers to the catabolic process involving the degradation of a cell's own components, such as long-lived proteins, protein aggregates, cellular organelles, cell membranes, organelle membranes, and other cellular components. The mechanism of autophagy may include: (i) the formation of a membrane around a targeted region of the cell, separating the contents from the rest of the cytoplasm, (ii) the fusion of the resultant vesicle with a lysosome and the subsequent degradation of the vesicle contents. The autophagy-misregulation associated disease may be a disease caused by misfolded protein aggregates, such as Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease. Also included are disease or disorder such as cancer, cardiovascular, neurodegenerative, metabolic, pulmonary, renal, infectious, musculoskeletal, and ocular disorders, in which the induction of autophagy can contribute to delaying the onset, slowing, stopping, or reversing the progression of one or more of symptoms associated with the disease or disorder. The autophagy-misregulation associated disease also includes cancer, e.g. any cancer in which the induction of autophagy would inhibit cell growth and division, reduce mutagenesis, remove mitochondria and other organelles damaged by reactive oxygen species or kill developing tumor cells. The term further means to include psychiatric diseases or disorders, e.g. any psychiatric disease or disorder in which the induction of autophagy would contribute to delaying the onset, slowing, stopping, or reversing the progression of one or more of symptoms associated with the psychiatric disease or disorder. In one embodiment, the psychiatric disease or disorder is selected from schizophrenia and bipolar disorder.

By some embodiments, the disease or condition associated with autophagy-misregulation is selected from Alzheimer's disease and cancer associated with reduced autophagic activity.

By some other embodiments, the disease or condition is a neurodegenerative disease, for example selected from Alzheimer's disease, Parkinson's disease, amyotrophic lateral sclerosis (ALS), multiple sclerosis, Huntington's disease, spinocerebellar ataxia, oculopharyngeal muscular dystrophy, multiple system atrophy Lewy body disease, and prion diseases.

As known, the effective amount for purposes herein may be determined by such considerations as known in the art. The amount must be effective to achieve the desired therapeutic effect, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, the effective amount depends on a variety of factors including a variety of pharmacological parameters such as half-life in the body, on undesired side effects, if any, on factors such as age and gender, and others.

The term treatment or any lingual variation thereof, as used herein, refers to the administering of a therapeutic amount of the formulations or preparations of the present disclosure which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

The term subject means to denote a mammal, human or non-human.

As used herein, the term about is meant to encompass deviation of ±10% from the specifically mentioned value of a parameter, such as temperature, concentration, etc.

Unless otherwise specifically indicated, all concentrations disclosed herein are provided as weight percentage, wt %, out of the weight of the formulation or the preparation, as the case may be.

Whenever a numerical range is indicated herein, it is meant to include any cited numeral (fractional or integral) within the indicated range. The phrases ranging/ranges between a first indicate number and a second indicate number and “ranging/ranges from” a first indicate number “to” a second indicate number are used herein interchangeably and are meant to include the first and second indicated numbers and all the fractional and integral numerals therebetween.

Unless the context requires otherwise, the word comprise, and variations such as “comprises” and “comprising”, will be understood to imply the inclusion of a stated integer or step or group of integers or steps but not the exclusion of any integer or step or group of integers and steps.

The term . . . at least one . . . as applied to any component of a formulation should be read to encompass one, two, three, four, or even more different occurrences of said component in the formulation or preparation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1F are pictures of exemplary formulations according to some examples of this disclosure.

FIGS. 2A-2C show the droplet size (Z-average) (●) and PDI (▪) measurement results as a function of the concentration of Compound GHF-201 in the formulation: 8CS-RG (FIG. 2A), LDS-C (FIG. 2B), LDS-600 (FIG. 2C).

FIGS. 3A-3C show the viscosity as a function of the concentration of Compound GHF-201 in the formulation: 8CS-RG (FIG. 3A), LDS-C (FIG. 3B), LDS-600 (FIG. 3C).

FIGS. 4A-4B shows the refractive index RI (FIG. 4A) and normalized RI (FIG. 4B) viscosity as a function of the concentration of Compound GHF-201 in the formulation: 8CS-RG (●), LDS-C (▪), LDS-600 (▴).

FIGS. 5A-5L are LUMiFuge test results for exemplary formulations loaded with Compound GHF-201: 8CS-RG vehicle (FIG. 5A), 8CS-RG 2.5% GHF-201 (FIG. 5B), 8CS-RG 5.0% GHF-201 (FIG. 5C), 8CS-RG 7.5% GHF-201 (FIG. 5D), LDS-C vehicle (FIG. 5E), LDS-C 2.5% GHF-201 (FIG. 5F), LDS-C 5.0% GHF-201 (FIG. 5G), LDS-C 7.5% GHF-201 (FIG. 5H), LDS-600 vehicle (FIG. 5I), LDS-600 2.5% GHF-201 (FIG. 5J), LDS-600 5.0% GHF-201 (FIG. 5K), LDS-600 7.5% GHF-201 (FIG. 5L).

FIGS. 6A-6C are photos of formulation LDS-C. 7.15% Compound GHF-201, at physical stability tests: at t=0 (FIG. 6A), after 12 months at 25° C. (FIG. 6B), and after 12 months at 40° C. (FIG. 6C).

FIGS. 7A-7C show DLS analysis results for formulation LDS-C, 7.15% Compound GHF-201, at physical stability tests: at t=0 (FIG. 7A), after 12 months at 25° C. (FIG. 7B), and after 12 months at 40° C. (FIG. 7C).

FIGS. 8A-8B show tissue penetration test results in APBD model mice for Compound GHF-201: non-formulated (FIG. 8A) and formulated in LDS-C formulation, 7.5 wt % (FIG. 8B).

FIGS. 9A-9C are pharmacokinetic profiles of Compound GHF-201 administered per os in formulation LDS-C to APBD patient 1 (FIG. 9A) and IV at 250 mg/kg to n=3 APBD modeling mice (FIG. 9B). Active ingredient doses in days 1, 2, and 3 were 170 mg, 255 mg, and 340 mg, respectively. Encircled in A, values blown up in FIG. 9C.

FIGS. 10A-10C are pharmacokinetic profiles of Compound GHF-201 administered per os in formulation LDS-C to APBD patient 2 (FIG. 10A) and IV at 250 mg/kg to n=3 APBD modeling mice (FIG. 10B). Active ingredient doses in days 1, 2, and 3 were 170 mg. 255 mg, and 340 mg, respectively. Values blown up in FIG. 10C.

FIGS. 11A-11B are Areas Under the Curve (AUC) at correlate with dosage administered in consecutive days for patients 1 and 2, respectively.

FIGS. 12A-12C show muscle power grade for patient 1 (before treatment and after 10 months of treatment) (FIG. 12A), and neurofilament light chain in plasma for patient 1 (FIG. 12B) and patient 2 (FIG. 12C), treated with a formulation of Compound GHF-201.

### DETAILED DESCRIPTION OF EMBODIMENTS

### Exemplary Formulations

Empty formulations were prepared by weighing all components according to Table 1, and mixing them at 40-60° C.

Compound of formula (IA), to be referred to herein as Compound GHF-201 (or alternatively as Compound A) was selected as exemplary compound:

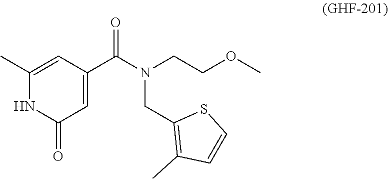

Compound GHF-201 was then solubilized into the formulation at various concentrations (2.5, 5.0, 7.5 wt %, weight percent of the weight of the empty formulation).

TABLE 1
Exemplary formulations (without Compound GHF-201)

| | | | LDS- | LDS- | LDS- | LDS- | LDS- | LDS- |
|---|---|---|---|---|---|---|---|---|
| Component | Function | LDS-C | C′a | C′b | C′c | C′d | C′e | C′f |
| Cremophor EL | Hydrophilic | 22.54 | — | 22.54 | 22.58 | 22.58 | 22.58 | 22.54 |
| HECO 40 | surfactants | — | — | 19.20 | — | — | — | — |
| Tween 80 | | 19.20 | 19.20 | — | 19.23 | 19.23 | 19.23 | 19.20 |
| Tween 20 | | — | 22.54 | — | — | — | — | — |
| Tween 60 | | — | — | — | — | — | — | — |
| Phosal 50 PG | Co- | 6.08 | 6.08 | 6.08 | 9.09 | 6.06 | 3.03 | 6.08 |
| Propylene glycol | surfactants | 12.00 | 12.00 | 12.00 | 17.95 | 11.97 | 5.98 | 12.00 |
| PEG 400 | | 10.00 | 10.00 | 10.00 | 14.96 | 9.97 | 4.99 | 10.00 |
| MCT | Oil | 2.18 | 2.18 | 2.18 | 2.19 | 2.19 | 2.19 | — |
| IPM | | — | — | — | — | — | — | — |
| Oleyl alcohol | | — | — | — | — | — | — | 2.18 |
| Ethanol | Solvent | 28.00 | 28.00 | 28.00 | 14.00 | 28.00 | 42.00 | 28.00 |

| | | | | | | | |
|---|---|---|---|---|---|---|---|
| Total | 100.0 | 100.0 | 100.0 | 100.0 | 100.0 | 100.0 | 100.0 |
| Total surfactants | 41.74 | 41.74 | 41.74 | 41.81 | 41.81 | 41.81 | 41.74 |
| Total co-surfactants | 28.08 | 28.08 | 28.08 | 42.00 | 28.00 | 14.00 | 28.08 |
| Total oils | 2.18 | 2.18 | 2.18 | 2.19 | 2.19 | 2.19 | 2.18 |
| Total solvents | 28.00 | 28.00 | 28.00 | 14.00 | 28.00 | 42.00 | 28.00 |

| | | 8CS- | 8CS- | LDS- | LDS- | LDS- | LDS- |
|---|---|---|---|---|---|---|---|
| Component | Function | RG | RG′a | 600 | 600′a | 600′b | 600′° C. |
| Cremophor EL | Hydrophilic | 24.54 | 24.54 | — | — | 28.52 | — |
| HECO 40 | surfactants | — | — | 0.90 | — | 0.90 | 28.52 |
| Tween 80 | | 19.80 | 19.80 | — | — | — | — |
| Tween 20 | | — | — | — | 0.90 | — | — |
| Tween 60 | | — | — | 28.52 | 28.52 | — | 0.90 |
| Phosal 50 PG | Co- | 6.08 | 6.08 | — | — | — | — |
| Propylene glycol | surfactants | 15.09 | 15.09 | 15.09 | 15.09 | 15.09 | 15.09 |
| PEG 400 | | — | — | 21.01 | 21.01 | 21.01 | 21.01 |
| MCT | Oil | 2.19 | — | 2.19 | 2.19 | 2.19 | 2.19 |
| IPM | | — | 2.19 | — | — | — | — |
| Oleyl alcohol | | — | — | — | — | — | — |
| Ethanol | Solvent | 32.30 | 32.30 | 32.30 | 32.30 | 32.30 | 32.30 |

| | | | | | | |
|---|---|---|---|---|---|---|
| Total | 100.0 | 100.0 | 100.0 | 100.0 | 100.0 | 100.0 |
| Total surfactants | 44.34 | 44.34 | 29.42 | 29.42 | 29.42 | 29.42 |
| Total co-surfactants | 21.17 | 21.17 | 36.10 | 36.10 | 36.10 | 36.10 |
| Total oils | 2.19 | 2.18 | 2.19 | 2.19 | 2.19 | 2.19 |
| Total solvents | 32.30 | 32.30 | 32.30 | 32.30 | 32.30 | 32.30 |

### Physical Characterization

The formulations (for example those of Table 1 above) are devoid of water and are fully dilutable in an aqueous liquid. During dilution, the formulation forms nanostructures, with an average droplet size of about 20 nm.

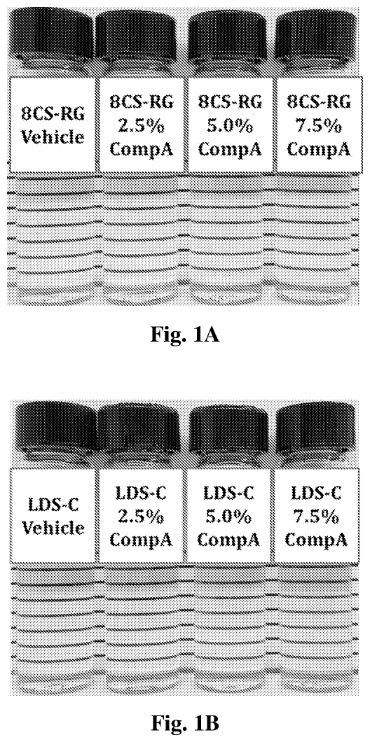

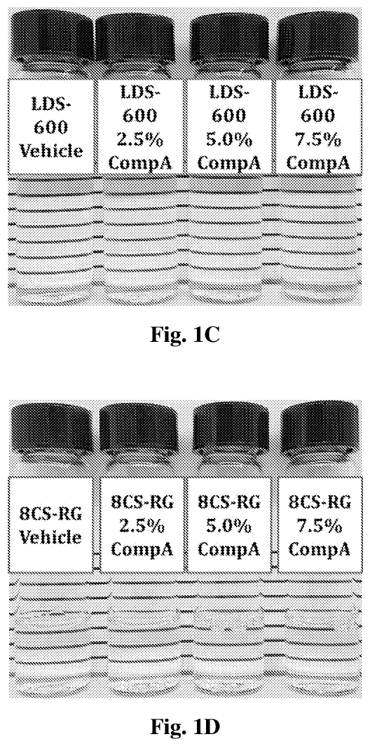

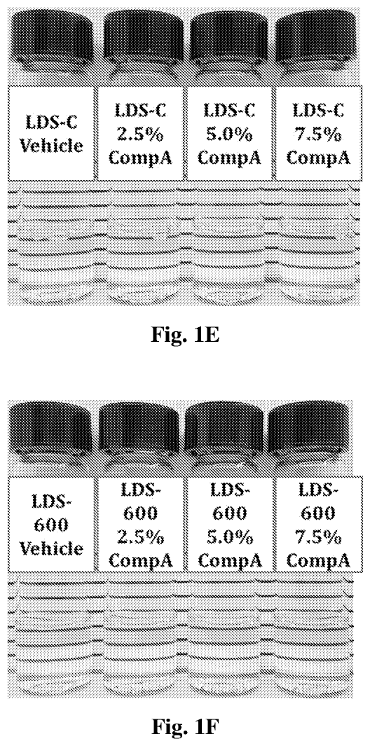

Selected formulations from Table 1 were analyzed to assess their physical characteristics. Exemplary visualization of the appearance of some of the formulations can be seen in FIGS. 1A-1F: the formulations in non-diluted form (FIGS. 1A-1C) and diluted (90% water content) (FIGS. 1D-1E), without Compound GHF-201 (“vehicle”) and loaded with 2.5 wt %, 5 wt % and 7.5 wt % of Compound GHF-201. As can be seen, all formulations, in both non-diluted and diluted forms have shown high transparency, homogeneity and thermodynamic stability.

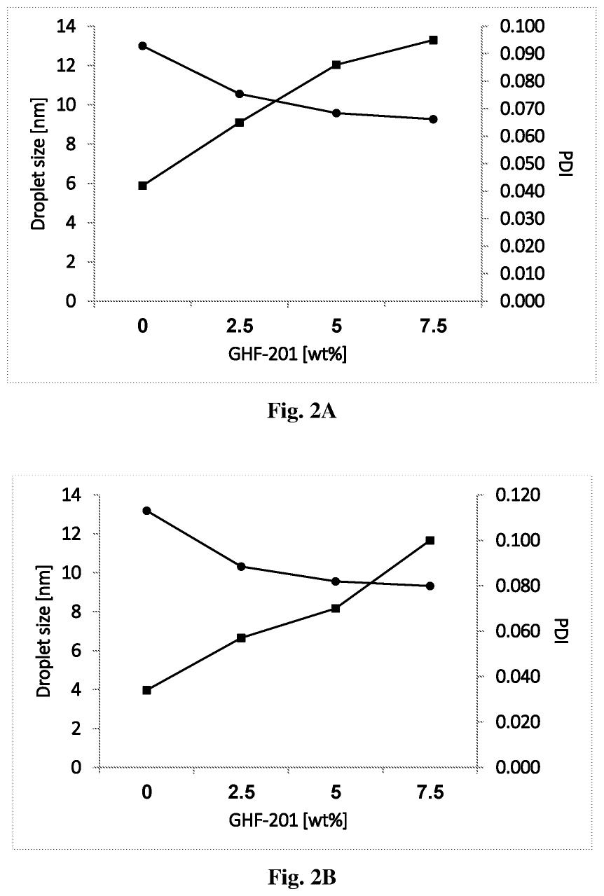

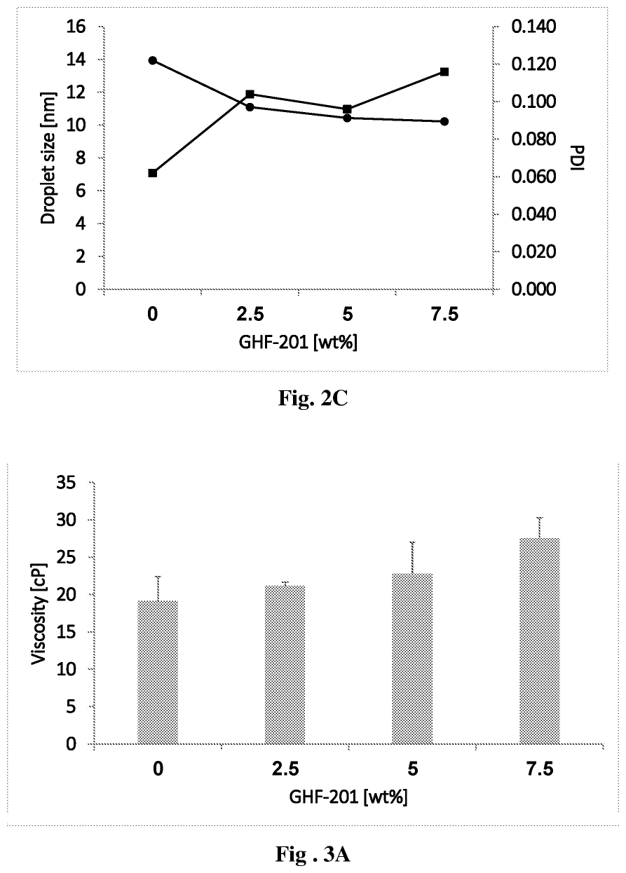

The hydrodynamic radii of the droplets were measured at room temperature by dynamic light scattering (DLS) using Nano-ZS Zetasizer (Malvern, UK), with water as a dispersant; results are shown in Table 2 and in FIGS. 2A-2C.

TABLE 2
Droplet size and PDI of selected formulations

| | % comp. | Droplet size (nm) | | PDI | |
|---|---|---|---|---|---|
| Formulation | GHF-201 | Average | SD | Average | SD |
| 8CS-RG | Vehicle | 13.00 | 0.20 | 0.042 | 0.006 |
| | 2.5 | 10.56 | 0.13 | 0.065 | 0.005 |
| | 5 | 9.574 | 0.112 | 0.086 | 0.005 |
| | 7.5 | 9.269 | 0.051 | 0.095 | 0.007 |
| LDS-C | Vehicle | 13.19 | 0.10 | 0.034 | 0.003 |
| | 2.5 | 10.32 | 0.05 | 0.057 | 0.006 |
| | 5 | 9.557 | 0.043 | 0.070 | 0.007 |
| | 7.5 | 9.319 | 0.047 | 0.100 | 0.008 |
| LDC-600 | Vehicle | 13.94 | 0.11 | 0.062 | 0.011 |
| | 2.5 | 11.10 | 0.05 | 0.104 | 0.010 |
| | 5 | 10.43 | 0.02 | 0.096 | 0.006 |
| | 7.5 | 10.22 | 0.04 | 0.116 | 0.006 |

As seen in Table 2 and FIGS. 2A-2C, increase in concentration and solubilization of Compound GHF-201 in the formulation caused the droplets to shrink and the PDI to slightly increase. For the empty formulations (vehicles), the average droplet size varied from 13 to 14 nm, while for the formulations loaded with Compound GHF-201 the droplet size was of about 10-11 nm and 9-10 nm for 2.5% and 5-7.5% Compound GHF-201, respectively. The measured PDI values were about 0.03-0.06 for the vehicles and up to 0.11 for 7.5% Compound GHF-201. Overall, for both vehicles and loaded formulations, the PDI values were low, indicating relatively monodisperses systems.

Without wishing to be bound by theory, the solubilization of Compound GHF-201 is hypothesized to result in stronger interactions of the co-surfactants and solvent at the interface of the droplets. By increasing Compound GHF-201 concentrations, more droplets of lower sized are formed, causing the gradual decrease in average droplet size and increase in the polydispersity.

To further understand the interactions between Compound GHF-201 and the excipients in the formulations, the viscosity and NMR measurements were carried out.

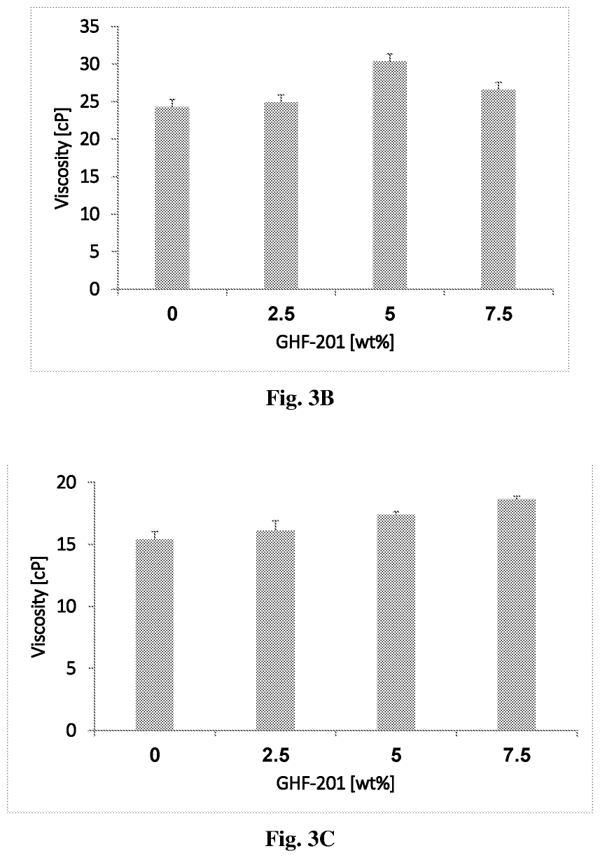

The viscosity measurements were conducted using RS6000 rheometer (Thermo Scientific) equipped with C60/1 TiL-L12007 cone, operated under rotational mode within shear rates range of 0.01000-100.0 l/s for 6 minutes. All measurements were conducted at 25±1° C. Under these conditions the formulations demonstrate Newtonian behavior, thus, the formulations can be characterized by their viscosity. The viscosities of the systems were calculated based on linear fit of the rotational shears (t) versus the shear rate (y). The slopes of the linear fit represent viscosity. Table 3 and FIGS. 3A-3C summarize the measured viscosities for the preconcentrates of 8CS-RG, LDS-C and LDS-600 systems.

TABLE 3
viscosity of selected formulations

| | |
|---|---|
| % comp. | Viscosity (cP) |

| Formulation | GHF-201 | Average | SD |
|---|---|---|---|
| 8CS-RG | Vehicle | 19.16 | 3.24 |
| | 2.5 | 21.16 | 0.49 |
| | 5 | 22.82 | 4.18 |
| | 7.5 | 27.55 | 2.72 |
| LDS-C | Vehicle | 24.29 | 2.25 |
| | 2.5 | 24.91 | 4.06 |
| | 5 | 30.35 | 0.62 |
| | 7.5 | 26.56 | 3.48 |
| LDC-600 | Vehicle | 15.40 | 0.64 |
| | 2.5 | 16.10 | 0.81 |
| | 5 | 17.40 | 0.23 |
| | 7.5 | 18.64 | 0.25 |

As demonstrated in FIGS. 3A-3C, the increase in Compound GHF-201 concentration resulted in an increase in the formulations' viscosity. Without wishing to be bound by theory, the presence of Compound GHF-201 in all examined formulations resulted in stronger interactions between the API and the excipients, mostly the co-surfactants, which contributes to a larger resistance of the formulations to applied shear. This indicates that introduction of compound GHF-201 into the formulation increases its stability and also its resistance to dynamic changes.

The pH and refractive index were measured for the three exemplary formulations. The results are summarized in Table 4. For pH measurements the samples were diluted 10 times with purified water; results in Table 4 are normalized to the pH of the water.

TABLE 4
normalized pH and RI values of selected formulations

| Formulation | % Comp. GHF-201 | Normalized pH* | Refractive index |
|---|---|---|---|
| 8CS-RG | Vehicle | 1.00 | 1.426 |
| | 2.5 | 1.01 | 1.429 |
| | 5 | 1.02 | 1.431 |
| | 7.5 | 1.02 | 1.434 |
| LDS-C | Vehicle | 1.04 | 1.432 |
| | 2.5 | 1.05 | 1.434 |
| | 5 | 1.05 | 1.434 |
| | 7.5 | 1.05 | 1.440 |
| LDC-600 | Vehicle | 0.82 | 1.425 |
| | 2.5 | 0.82 | 1.428 |
| | 5 | 0.82 | 1.431 |
| | 7.5 | 0.82 | 1.435 |

*pHₛₐₘₚₗₑ/pH_{water}

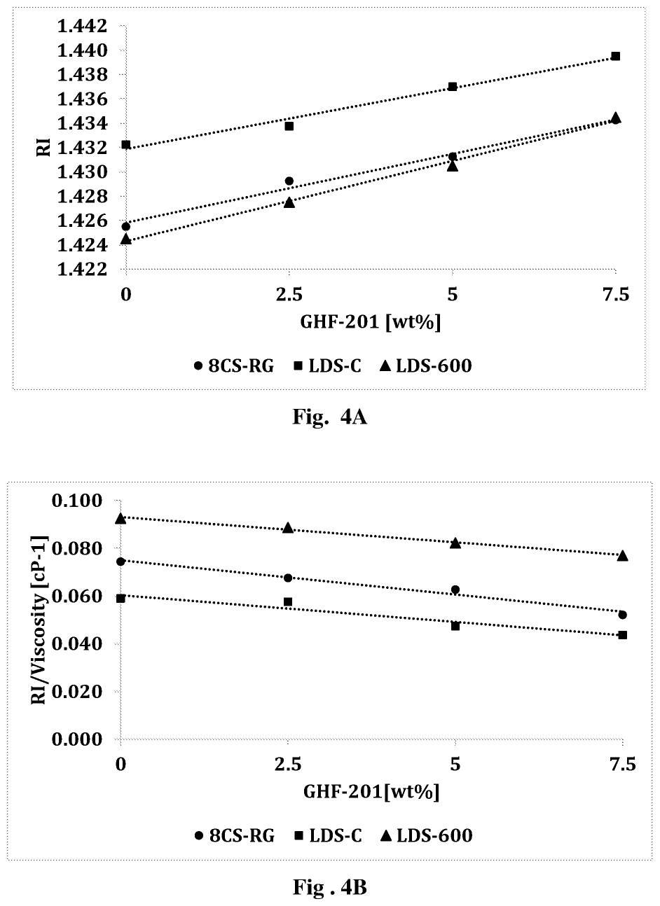

As demonstrated by Table 4, the pH was not affected by the solubilization of Compound GHF-201. As for the refractive index (RI), it shows linear behavior as a function of Compound GHF-201 concentration with similar slopes of about 0.001 (1%) for all systems. It is noted that the RI is affected by the viscosity of the system, so as the viscosity increases the RI increases as well. In this case, the viscosity also increases with the increase in Compound GHF-201 content, so this trend in the RI as a function of Compound GHF-201 concentration is due to the viscosity. Interestingly, when normalizing the RI by the viscosity, the resulting quotient decreases as a function of Compound GHF-201's concentration (see FIGS. 4A-4B). Without wishing to be bound by theory, these results may be attributed to the interface of the nanostructures which becomes more “saturated” by Compound GHF-201 and thus affects the refraction of light.

The mobility of the systems' components was also measured by PGSE-NMR. The diffusion coefficients (×10-11) of several excipients and excipients groups are presented in Tables 5-1 to 5-3. Measurements were carried out under gradient/pulses program for which: δ=10.0 ms, Δ=27.0, gmax=44.79. D value for the surfactants and oil is a mean calculated for the two surfactants in each formulation and MCT.

TABLE 5-1
diffusion coefficients (×10⁻¹¹) of components of 8CS-RG in both
concentrated and diluted forms, with and without Compound GHF-201

| | | Diffusion coefficient ×10⁻¹¹ (m²/s) | | | | |
|---|---|---|---|---|---|---|
| | | Surfac- | | | | |
| Water | Comp.A | tants + | | | Comp. | |
| (wt %) | (wt %) | MCT | PG | EtOH | GHF201 | Water |
| 0 | 0 | 2.16 | 17.03 | 28.57 | | |
| | 2.5 | 1.99 | 16.03 | 27.43 | 5.03 | |
| | 5 | 1.97 | 12.91 | 24.70 | 4.52 | |
| | 7.5 | 1.75 | 12.43 | 23.67 | 3.89 | |
| 90 | 0 | 2.18 | 63.10 | 76.80 | | 150.00 |
| | 2.5 | 2.20 | 62.09 | 76.20 | 24.19 | 148.00 |
| | 5 | 2.23 | 62.39 | 78.43 | 22.15 | 152.00 |
| | 7.5 | 2.24 | 63.61 | 74.47 | 20.35 | 150.00 |

TABLE 5-2
diffusion coefficients (×10⁻¹¹) of components of LDS-C in both
concentrated and diluted forms, with and without Compound GHF-201

| | | Diffusion coefficient ×10⁻¹¹ (m²/s) | | | | |
|---|---|---|---|---|---|---|
| | | Surfac- | | | | |
| Water | Comp.A | tants + | | | Comp. | |
| (wt %) | (wt %) | MCT | PG | EtOH | GHF201 | Water |
| 0 | 0 | 2.16 | 17.03 | 28.57 | | |
| | 2.5 | 1.99 | 16.03 | 27.43 | 5.03 | |
| | 5 | 1.97 | 12.91 | 24.70 | 4.52 | |
| | 7.5 | 1.75 | 12.43 | 23.67 | 3.89 | |
| 90 | 0 | 2.18 | 63.10 | 76.80 | | 150.00 |
| | 2.5 | 2.20 | 62.09 | 76.20 | 24.19 | 148.00 |
| | 5 | 2.23 | 62.39 | 78.43 | 22.15 | 152.00 |
| | 7.5 | 2.24 | 63.61 | 74.47 | 20.35 | 150.00 |

TABLE 5-3
diffusion coefficients (×10⁻¹¹) of components
of LDS-600 system in both concentrated and diluted
forms, with and without Compound GHF-201

| | | Diffusion coefficient ×10⁻¹¹ (m²/s) | | | | | |
|---|---|---|---|---|---|---|---|
| | | Surfac- | | | | | |
| Water | Comp.A | tants + | | | PEG | Comp. | |
| (wt %) | (wt %) | MCT | PG | EtOH | 400 | GHF201 | Water |
| 0 | 0 | 3.26 | 19.69 | 30.23 | 25.53 | | |
| | 2.5 | 3.18 | 17.15 | 29.00 | 26.00 | 5.30 | |
| | 5 | 3.12 | 14.77 | 26.50 | 23.48 | 4.91 | |
| | 7.5 | 2.82 | 12.51 | 25.17 | 15.30 | 4.30 | |
| 90 | 0 | 2.67 | 60.53 | 77.07 | 65.80 | | 149.00 |
| | 2.5 | 2.61 | 58.17 | 76.67 | 54.82 | 24.93 | 148.00 |
| | 5 | 2.94 | 68.73 | 74.57 | 69.30 | 25.21 | 151.00 |
| | 7.5 | 2.92 | 62.56 | 79.40 | 64.58 | 23.98 | 149.00 |

Tables 5-1, 5-2 and 5-3 summarize the diffusion coefficient (D) of the surfactants and oil (MCT) and each individual diffusion coefficient of PG, EtOH, Compound GHF-201 and water. In case of LDS-600, the diffusion coefficient of PEG 400 was also calculated. Overall, in all tested formulations a clear trend of a decrease in the diffusivity of all tested components as a function of Compound GHF-201 concentration in the concentrate form was observed, suggesting that the gradual loading of Compound GHF-201 consistently results in tightened interactions between all the components. Remarkably, when the samples are being diluted, the diffusivity of Compound GHF-201 decreases while the mobility of the surfactants increases in all tested systems. This indicated that Compound GHF-201 has a significant role as a structure builder in the systems that helps stabilize the nanodroplets in the presence of water. This also supports the observations from DLS measurements where the droplets progressively shrink as the content of Compound GHF-201 increases. The mobility of the solvent and co-surfactants in the diluted systems on the other hand, remains constant suggesting that the role of these components is to stabilize the concentrates rather than the diluted systems.

### Long-Term Physical Stability

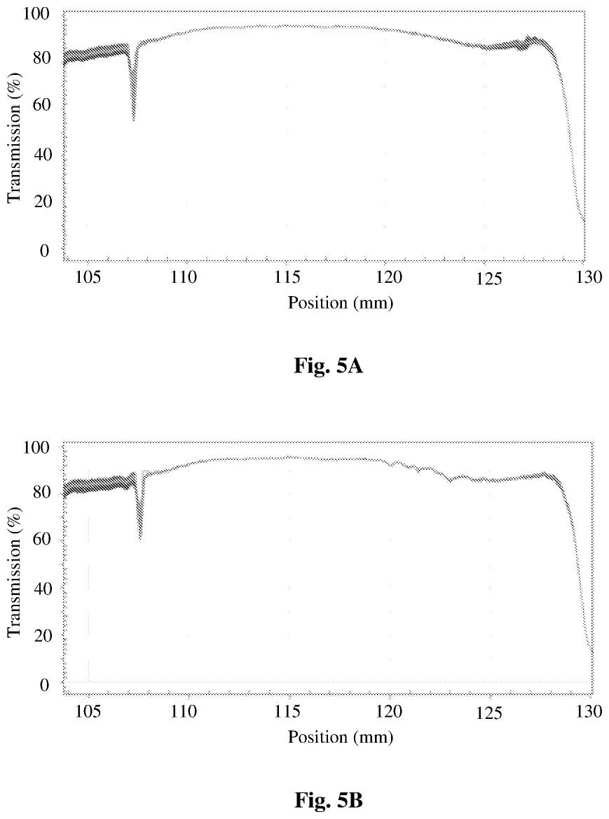

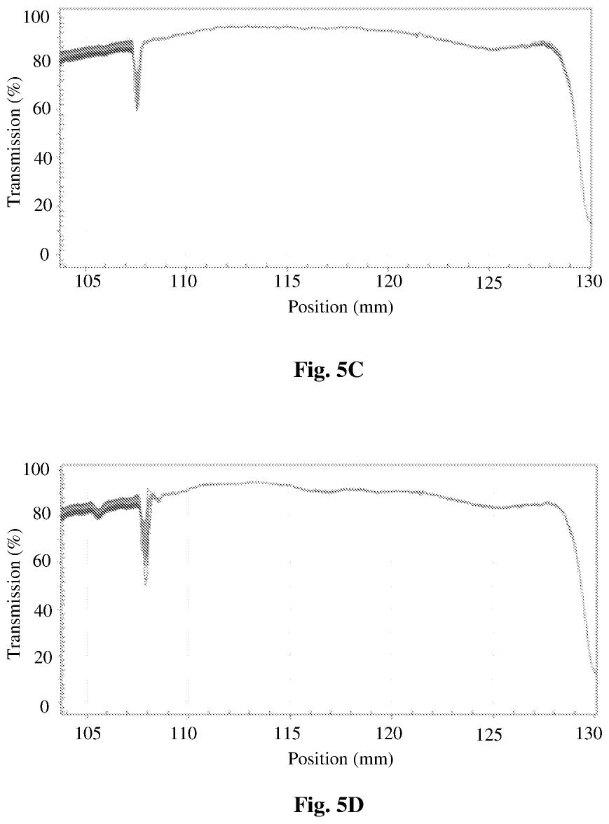

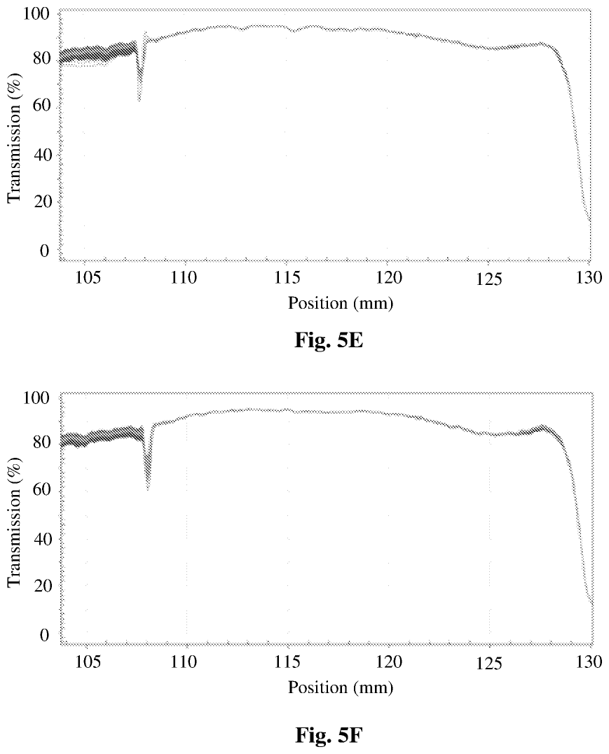

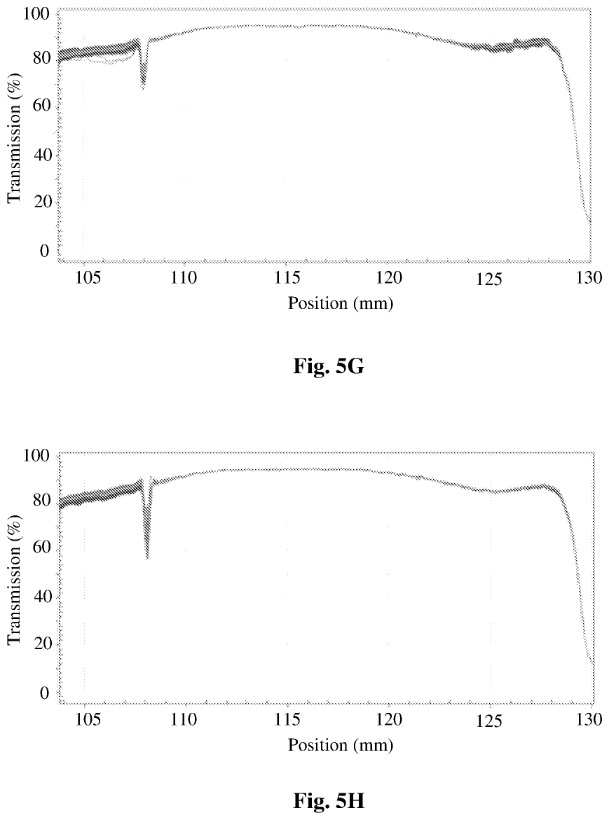

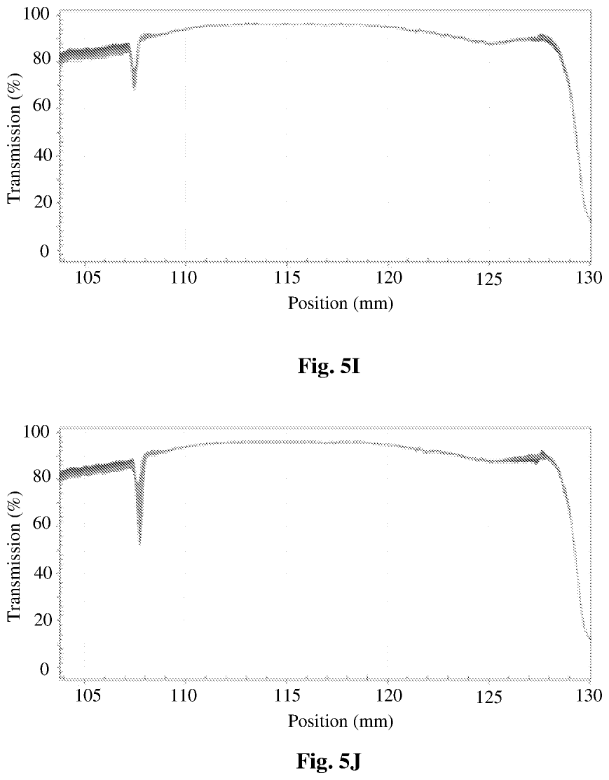

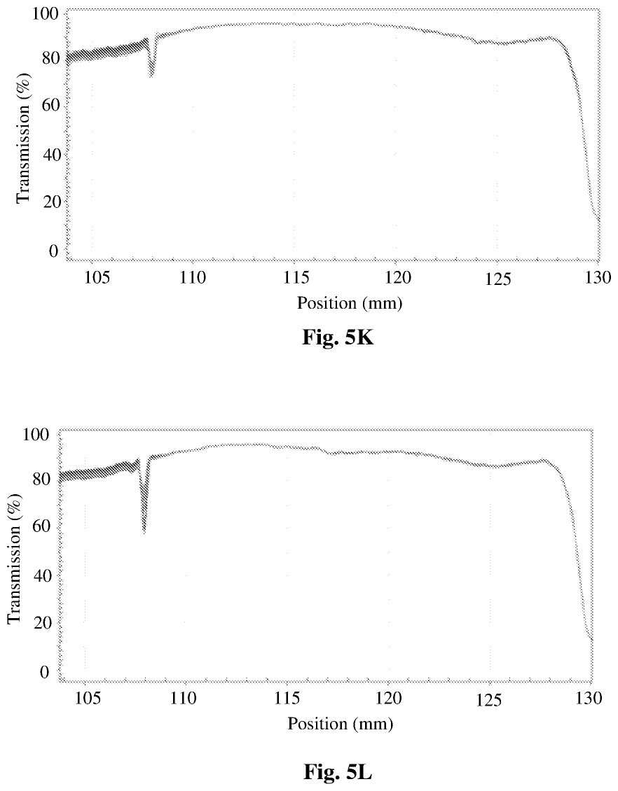

To determine long term stability of formulations, a rapid measurement was carried out using LUMiSizer® analytical centrifugation. The results are shown in FIGS. 5A-5L. LUMiSizer® analysis enables to predict the shelf-life of a formulation in its original concentration, even in cases of slow destabilization processes like sedimentation, flocculation, coalescence and fractionation. During LUMiSizer® measurements, parallel light illuminates the entire sample cell in a centrifugal field; the transmitted light is detected by sensors arranged linearly along the total length of the sample-cell. Local alterations of particles or droplets are detected due to changes in light transmission over time. The results are presented in a graph plotting the percentage of transmitted light (Transmission %) as a function of local position (mm), revealing the corresponding transmission profile over time.

The changes in transmission indicate the stability of the formulation-when the transmission profile remains constant, the samples are considered physically stable and their shelf-life can be extrapolated based on the measurement conditions.

As shown in FIGS. 5A-5L, in all transmission profiles the lines overlap, suggesting that no changes in the transmission were observed, and all the formulations are physically stable and expected, on the basis of this analysis, to be stable under storage conditions for at least 2 years.

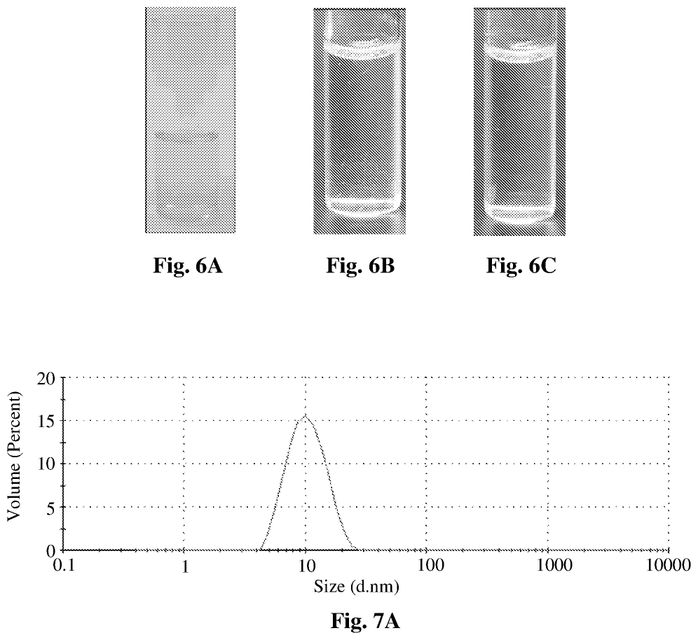

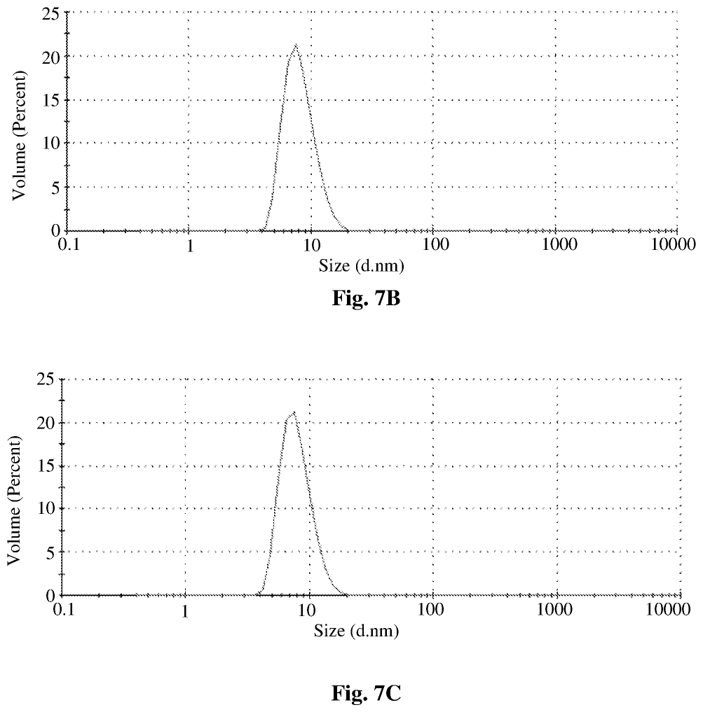

Physical stability was also assessed for formulation LDS-C with 7.15 wt % of Compound GHF-201 for 12 months at 25° C. and 40° C. The stability test results are provided in Tables 6-1 and 6-2. The visual appearance of the samples is shown in FIGS. 6A-6C, while DLS analysis results are shown in FIGS. 7A-7C.

TABLE 6-1
physical stability test results, 25° C., 12 months

| | Acceptance | | | | | | |
|---|---|---|---|---|---|---|---|
| | criteria | t₀ | t₁ | t₃ | t₆ | t₉ | t₁₂ |
| Appearance | Clear, | Clear, | Clear, | Clear, | Clear, | Clear, | Clear, |
| | oily | oily | oily | oily | oily | oily | oily |
| | liquid | liquid | liquid | liquid | liquid | liquid | liquid |
| Assay | 64.4-78.7 | 74.1 | 76.3 | 72.8 | 72.1 | 71.8 | 74.5 |
| (HPLC) | (90-110%) | (103.6%) | (105.2%) | (101.8%) | (100.9%) | (100.4%) | (104.2%) |
| [mg/g] | | | | | | | |
| Impurities | Unknown | N/A | N/A | <0.05% | <0.05% | <0.05% | <0.05% |
| | <0.5% | | | | | | |
| pH | 5-8 | 6.24 | 6.13 | 6.19 | 6.24 | 6.23 | 6.38 |
| Refractive | 1.435-1.450 | 1.441 | 1.439 | 1.440 | 1.439 | 1.441 | 1.441 |
| index | | | | | | | |
| Droplet | 7-15 | 9.6 | 9.67 | 9.54 | 8.98 | 9.04 | 9.50 |
| size (nm) | | | | | | | |
| PDI | 0.050-0.250 | 0.107 | 0.042 | 0.047 | 0.077 | 0.056 | 0.069 |

TABLE 6-2
physical stability test results, 40° C., 12 months

| | Acceptance | | | | | | |
|---|---|---|---|---|---|---|---|
| | criteria | t₀ | t₁ | t₃ | t₆ | t₉ | t₁₂ |
| Appearance | Clear, | Clear, | Clear, | Clear, | Clear, | Clear, | Clear, |
| | oily | oily | oily | oily | oily | oily | oily |
| | liquid | liquid | liquid | liquid | liquid | liquid | liquid |
| Assay | 64.4-78.7 | 74.1 | 74.6 | 72.9 | 70.3 | 72.0 | 75.7 |
| (HPLC) | (90-110%) | (103.6%) | (104.3%) | (102.7%) | (98.3%) | (100.7%) | (105.9%) |
| [mg/g] | | | | | | | |
| Impurities | Unknown | N/A | N/A | <0.05% | <0.05% | <0.05% | <0.05% |
| | <0.5% | | | | | | |
| pH | 5-8 | 6.24 | 6.13 | 6.15 | 6.03 | 6.03 | 6.07 |
| Refractive | 1.435-1.450 | 1.441 | 1.439 | 1.440 | 1.439 | 1.441 | 1.441 |
| index | | | | | | | |
| Droplet | 7-15 | 9.60 | 9.67 | 9.54 | 8.99 | 9.11 | 9.17 |
| size (nm) | | | | | | | |
| PDI | 0.050-0.250 | 0.107 | 0.042 | 0.047 | 0.072 | 0.061 | 0.051 |

As evident from the test results, the formulations are physically stable at storage temperatures for at least 12 months, without evidence of significant change in physical properties.

### Tissue Penetration

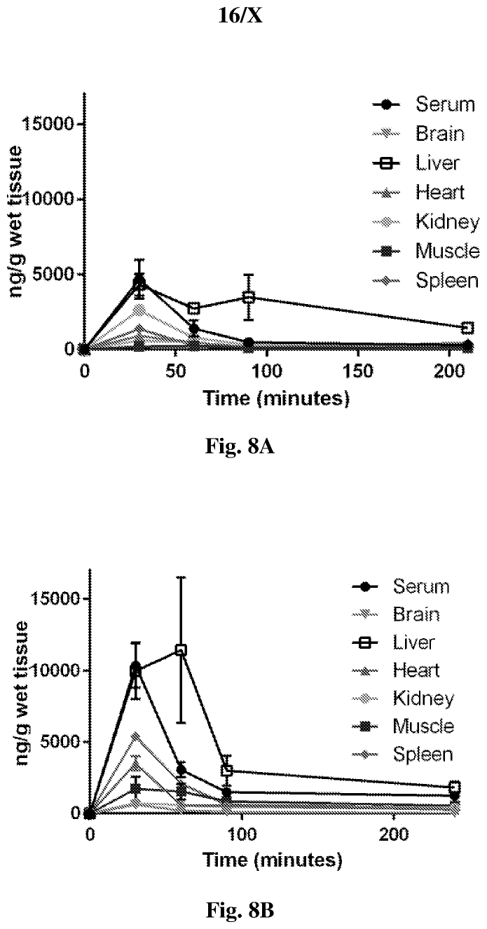

FIGS. 8A-8B show tissue penetration test results in APBD model mice for Compound GHF-201 in non-formulated form (FIG. 8A) and formulated in LDS-C formulation (FIG. 8B). For pharmacokinetic analysis, 100 μl serum as well as brain, kidney, hind limb quad muscle, heart, liver, and spleen tissues were collected, homogenized, and extracted with acetonitrile following established guidelines (Kapetanovic et al, 2006). Calibration curves were made with 0, 1, 10, 100, and 1,000 ng/ml GHF-201 in 1 mg/ml solutions of 4-tert-butyl-2-(4H-1,2,4-triazol-4-yl) phenol (ChemBridge) as internal standard (IS). Tissue samples were then dissolved in 1 mg/ml IS solutions and spiked with 0-1,000 ng/ml GHF-201 to generate standard curves from which tissue levels of GHF201 were determined. Samples were analyzed by the LC-MS/MS Sciex Triple Quad™ 5500 mass spectrometer.

Gbeys/ys mice injected subcutaneously with 250 mg/kg GHF-201 were sacrificed 30, 60, 90, and 210 min post injection, and the indicated tissues were removed, as well as 100 μL of serum drawn. Graph shows means (±SEM) of GHF201 levels in the different tissues determined by LC-MS/MS. Results obtained from n=3 mice at each time point.

The distribution and kinetic parameters of GHF-201 was assessed in different tissues. A comparison between FIG. 8A and FIG. 8B shows that the bioavailability of GHF-201 orally administered in a formulation according to this disclosure was increased at least three-fold in all tissues compared to subcutaneous injections of GHF-201 in solution.

### Pharmacokinetic Tests

Adult Polyglucosan Body Disease (APBD) is a rare, neurodegenerative disease most often affecting adults from an Ashkenazi Jewish origin. APBD is characterized by partial deficiency of Glycogen Branching Enzyme 1 (GBE1) activity, most commonly caused by mutational substitution of tyrosine 329 with a serine residue (Y329S). This loss of function mutation in GBE1 leads to the generation of aggregates of mal-constructed glycogen called “polyglucosan bodies” (PB, amylopectin-like polysaccharides with fewer branch points). Being out of solution and aggregates, PB cannot be digested by the glycogen digesting enzyme, glycogen phosphorylase. The amassing aggregates often lead to liver failure and death in childhood (Andresen's disease; glycogenosis type IV; GSDIV). Milder mutations of GBE1, such as p.Y329S, result in smaller PB, which do not disturb hepatocytes and most other cell types, merely accumulating in the sides of cells. In neurons and glia cells, however, these PBs plug the tight confines of axons over time, and lead to the debilitating and fatal progressive axonopathic disease APBD, which is often misdiagnosed with amyotrophic lateral sclerosis or multiple sclerosis. The PB aggregates in APBD may cause different phenotypical alterations, such as neurogenic bladder, partial motor dysfunction in extremities, sensorial dysfunction in the lower part of the body, and in some cases, cognitive impairment. The advances stages of the disease are characterized by difficulty in walking, impaired balance, progressive weakness, and can even lead to death. Currently, there is no standard of care for this condition.

Compound GHF-201 was discovered to be capable of reducing polyglucosans in APBD patient-derived skin fibroblasts. Meeting strict Absorption, Distribution, Metabolism, Excretion and Toxicity (ADMET) criteria in silico, Compound GHF-201 was found to be safe in mice (14-day study). Pharmacokinetically, subcutaneously injected in mice, compound GHF-201 had high dwell time and persistence (>3 h) in the liver, intermediate levels and persistence in brain and heart (1 h) and negligible distribution to the muscle. This pharmacokinetic profile matched the histopathological effect of Compound GHF-201 on the respective tissues with polyglucosans being most lowered in the liver, intermediately lowered in the brain and heart and not affected in the muscle. Continuous treatment of APBD mice with compound GHF-201 for two months prior to disease expected onset significantly increased survival and improved locomotive and reflex parameters. The ameliorative effect on extension reflex is especially important since the patient correlate is pyramidal tetraparesis or upper motor neuron signs which are one of the main neurological deficiencies in APBD patients.

Compound GHF-201 was administered to APBD modeling mice as a 5% DMSO solution, IV injected twice a week at a dose of 250 mg/kg, equivalent to a daily dose of 70 mg/kg. Daily injection was avoided due to the relatively long duration of treatment (6 months), which might have led to excessive scarring. Following positive results in the murine model, compound GHF-201 was administered to two APBD patients as a part of a 3-day oral dose escalation study, during which clinical safety and pharmacokinetic profiles were determined.

Blood was drawn during three consecutive days according to the following regime—pre-administration and 0.5 h, 1 h. 1.5 h, 2 h, and 4 h post-administration. Compound GHF-201 was administered as formulation LDS-C. 7.15% Compound GHF-201. Active ingredient doses were 170 mg on the first day, 255 mg on the second day, and 340 mg on the third day.

Following blood drawing, plasma was separated by centrifugation (up to 1 h from drawing) at 2000 g for 10 min. at 4° C. Compound GHF-201 levels in plasma (supernatant) samples were determined by LC-Mass Spectrometry (LC-MS/MS) using Sciex Triple Quad™ 5500 mass spectrometer. 100 μl of plasma were collected and extracted in 500 μl acetonitrile solution (acetonitrile/water 1:1 v/v). Calibration curves were made with 0.1, 10, 100, and 1000 ng/ml compound GHF-201 in 1 mg/ml solutions of 4-tert-butyl-2-(4H-1,2,4-triazol-4-yl) phenol (ChemBridge) as internal standard (IS). The samples were dissolved in 1 mg/ml IS solutions and spiked with 0-1000 ng/ml of compound GHF-201 to generate standard curves from which plasma levels of compound GHF-201 were determined.

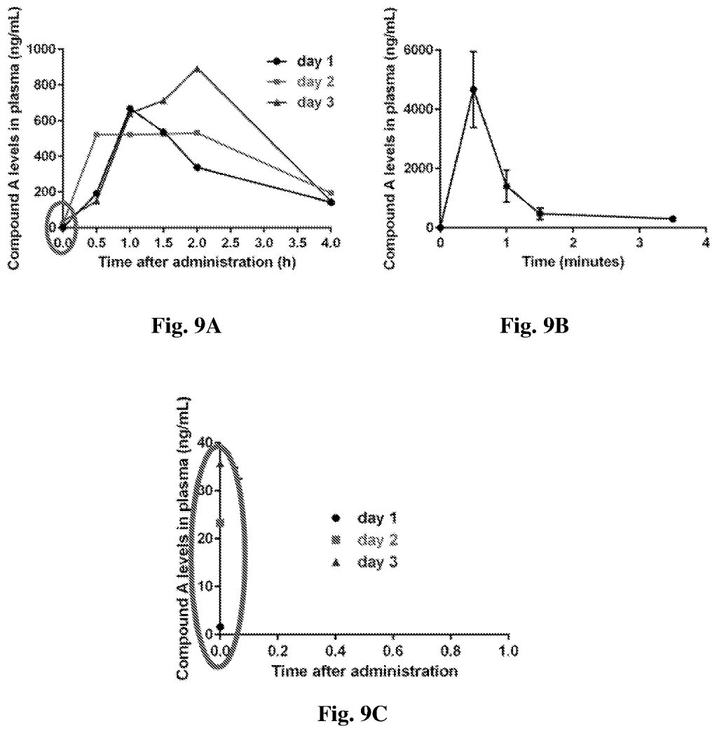

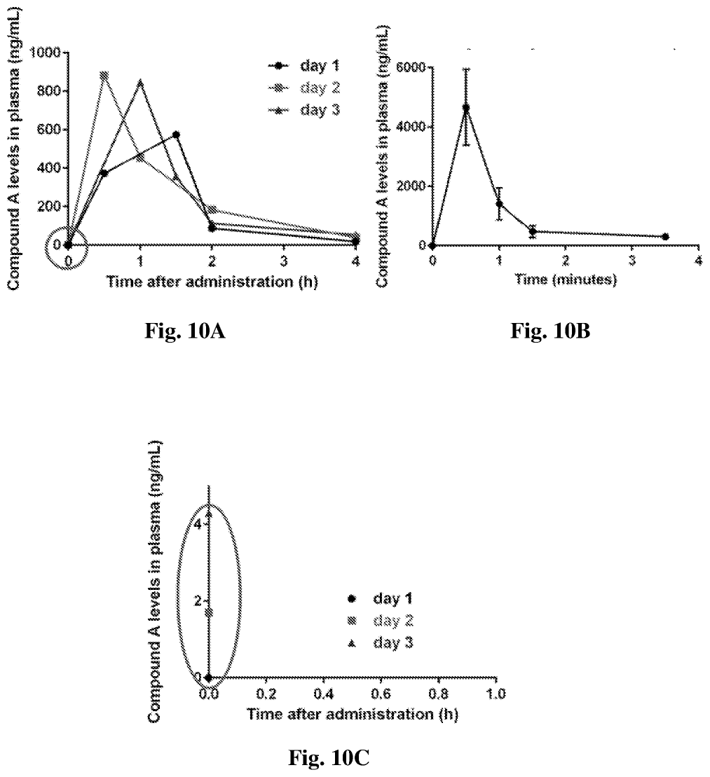

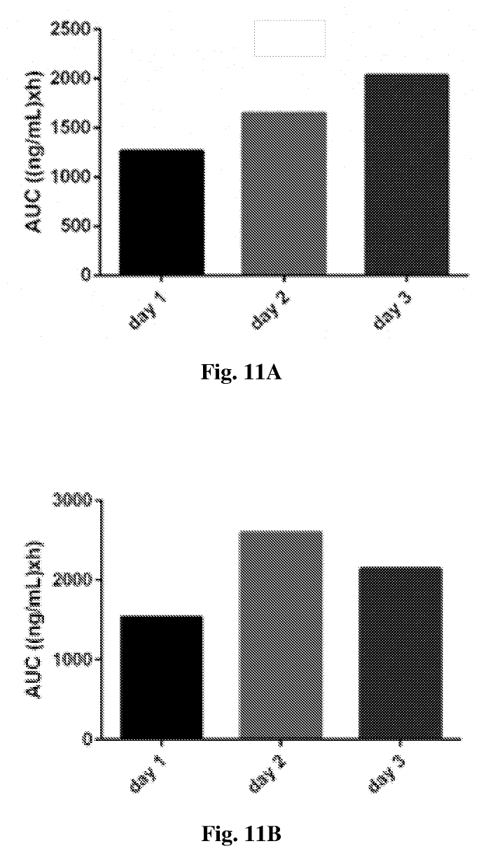

Patient 1's pharmacokinetic profile is shown compared to the mouse model profile in FIGS. 9A-9C, while the comparative pharmacokinetic profiles of Patient 2 are shown in FIGS. 10A-10C.

Following the FDA human equivalent dose calculation, mouse dose can be converted to human dose by dividing by 12. Cmax in mice was 4,700 ng/ml (FIGS. 9B, 10B). Divided by 12, 4,700 ng/mL yields 392 ng/mL. Cmax in patient 1 was 894 ng/ml (FIG. 9A) and in patient 2 was 883 ng/ml (FIG. 10A). It was found that, in mice, the administered dose produced a significant therapeutic effect. Therefore, it was assumed that a human equivalent, or higher, dose in patients is at least as effective as the equivalent dose in mice. Another assumption was that Cmax observed following administration of a therapeutic dose correlates with biological activity in the same way as the administered dose, these results can predict therapeutic efficacy in APBD patients.

In addition, it can be observed that administration of LDS-C formulation compound in patients delays time to Cmax, suggesting a “sustained release” mode of delivery. Time to Cmax increased with the increased administered dose Another interesting observation was that the pre-administration levels of compound GHF-201 in the plasma increased every consecutive day (FIGS. 9C, 10C), suggesting that a residual dose remained at the patient's plasma before the administration of the next dose in the following day and that these doses accumulated.

Importantly, Areas Under the Curve (AUC) correlated with the level of compound GHF-201 administered (FIGS. 11A-11B). This observation demonstrates that body exposure to the drug positively correlated with the level of drug administered. AUC was calculated as a sum of trapezoids rather than using integration and extrapolation to infinite time.

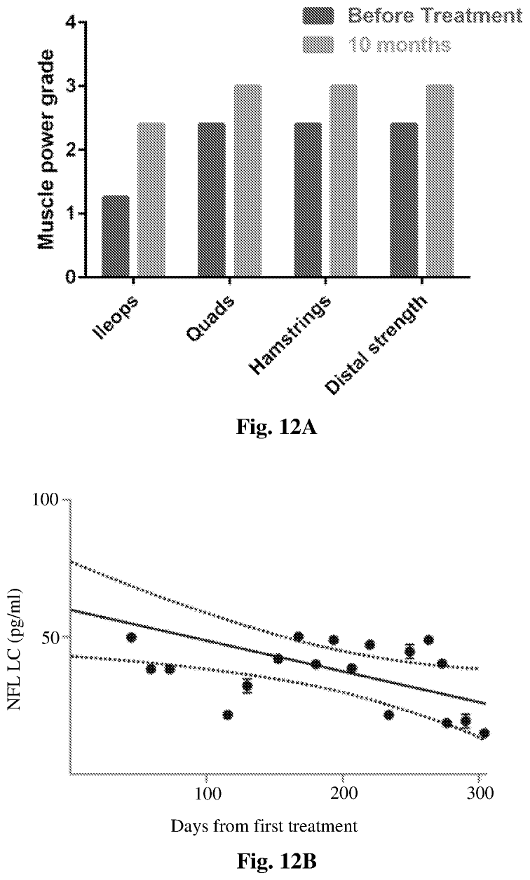

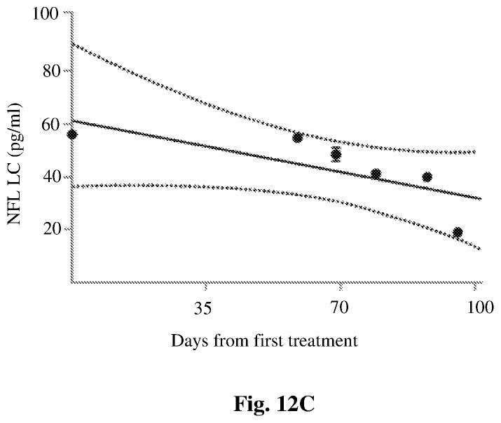

FIG. 12A shows muscle power grade for patient 1 (before treatment and after 10 months of treatment) (FIG. 12A), carried out according to the method described in Kleyweg et al. 1991. As can be seen, treatment led to a significant increase of muscle power in all muscles measured.

In FIGS. 12B-12C neurofilament light chain in plasma for patient 1 (FIG. 12B) and patient 2 (FIG. 12C), treated with a formulation of Compound GHF-201 are shown. The NFL level was obtained by analyzing patient plasma samples using the Simoa machine with the NF-Light v2 Advantage HD-X kit for the determination of human light chain neurofilament protein.

The reduction in neurofilament light chain levels indicates amelioration of neurodegeneration. Neurofilament light chains are considered a systemic biomarker for the extent of neurodegeneration in several neurodegenerative disorders such as ALS and AD. Administration of GHF-201 formulated in a formulation according to this disclosure showed significant decline in neurofilament light chains, suggesting amelioration of the extent of neurodegeneration.

### Further Exemplary Formulations

Empty formulations were prepared by weighing all components according to Table 7, and mixing them at 40-60° C.

Compound of formula (II), to be referred to herein as Compound GHF-205 was used for the additional formulations:

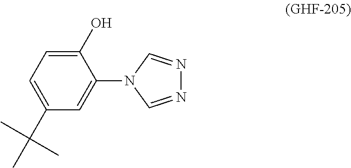 [SMILES: CC(C)(C)c1ccc(O)c(-n2cnnc2)c1]

TABLE 7
Exemplary formulation (without Compound GHF-205)

| Component | Function | RAG 204(250) | LDS-C(250) |
|---|---|---|---|
| HECO 40 | Hydrophilic | 0.90 | — |
| Cremophor EL | surfactants | — | 22.54 |
| Tween 80 | | 29.58 | 19.20 |
| Tween 60 | | 39.52 | — |
| Propylene glycol | Co-surfactants | 10.00 | 12.00 |
| PEG 400 | | — | 10.00 |
| Phosal 50 PG | | — | 6.08 |
| MCT | Oil | | 2.18 |
| Ethanol | Solvent | 20.00 | 28.00 |

| | | |
|---|---|---|
| Total | 100.0 | 100.0 |
| Total surfactants | 70 | 41.74 |
| Total co-surfactants | 10 | 28.08 |
| Total oils | 0 | 2.18 |
| Total solvents | 20 | 28.00 |

Compound GHF-205 was solubilized into the formulations at various concentrations (2.5 and 5.0 wt %). The formulations were tested for visual appearance, refractive index and droplet size, as detailed in Table 8.

TABLE 8
Physical characterization of formulations of GHF-205

| | GHF-205 | | Refrac- | Droplet | |
|---|---|---|---|---|---|
| | conc. | | tive | size | |
| Formulation | (wt %) | Appearance | index | (nm) | PDI |
| RAG204(205) | 0 | Clear, transparent | 1.4430 | 9.63 | 0.045 |
| | 2.5 | Clear, transparent | 1.4435 | 9.64 | 0.050 |
| | 5.0 | Clear, transparent | 1.4475 | 9.76 | 0.122 |
| LDS-C(205) | 0 | Clear, transparent | 1.4320 | 12.9 | 0.028 |
| | 2.5 | Clear, transparent | 1.4340 | 13.36 | 0.058 |
| | 5.0 | Clear, transparent | 1.4360 | 13.43 | 0.076 |

SD-NMR characterization was also carried out for LDS-C(205), loaded with 2.5 and 5 wt % of GHP-205, in concentrate form., as detailed in Table 9.

TABLE 9
Diffusion coefficients of components in formulation
LDS-C(205) (presented in ×10⁻¹¹ m²/s)

| | GHF-205 | | | | |
|---|---|---|---|---|---|
| | conc. | Surfactants + | Co- | | |
| Formulation | (wt %) | MCT* | surfactants | Ethanol | GHF-205 |
| LDS-C(205) | 0 | 1.93 | 16.24 | 24.50 | — |
| | 2.5 | 1.76 | 18.66 | 23.50 | 3.88 |
| | 5.0 | 1.87 | 15.12 | 18.0 | 3.36 |
| LDS-C(205), | 0 | 1.97 | 62.47 | ND | — |
| 90% water | | | | | |
| dilution | | | | | |

*The diffusivity of MCT was not distinguishable from the surfactants

In general, one can see that the co-surfactants in the concentrated system diffuse faster (higher mobility) compared to the surfactant. This suggests that while the hydrophilic surfactants and the lipophilic components form the main building blocks of the structure's interface in the concentrate form-once diluted, the lipophilic components are no longer needed to stabilize the interface (as their mobility is higher in the diluted form compared to the concentrate form). Thus, in the concentrate form, the lipophilic components are located at the interface and are essential in building the droplets' structure; upon dilution the lipophilic components move away from the interface and are located nearer to the external phase. This indicates that upon oral intake, the structural change will enable GHF-205 to migrate out of the formulation.

1.-37. (canceled)

38. A pharmaceutical formulation for oral delivery of a compound of formula (I) or a pharmaceutically acceptable salt, isomer or tautomer thereof, 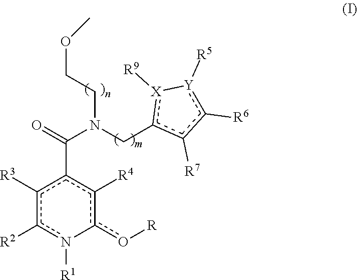 [symbol]
represents a single or a double bond,
n and m are integers, each being independently selected the group consisting of 1, 2 or 3;
R and R¹ are each independently hydrogen or is absent;
R², R³, R⁴, R⁵, R⁶, R⁷, R⁸ and R⁹, are each hydrogen, or are each being independently selected from the group consisting of alkyl, cycloalkyl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, and sulfonamide, each being further substituted or non-substituted; and
one of X and Y is S, while the other of X and Y is C; provided that when X is S then R⁹ is absent, and when Y is S then R⁵ is absent;
wherein the formulation comprises:
a) said compound of formula (I) or a pharmaceutically acceptable salt, isomer or tautomer thereof;
b) at least one hydrophilic surfactant in a total amount ranging between about 10 wt % and about 50 wt %;
c) at least one solvent in a total amount of at least about 20 wt %;
d) at least one co-surfactant; and
e) at least one oil in an amount of between 0 wt % and about 5 wt % of the formulation;
the weight ratio of said at least one solvent to said at least one hydrophilic surfactant ranges between about 1.25:1 and about 1:3.

39. The formulation of claim 38, wherein the compound of formula (I) is a compound of formula (l′) or a pharmaceutically acceptable salt, isomer or tautomer thereof, 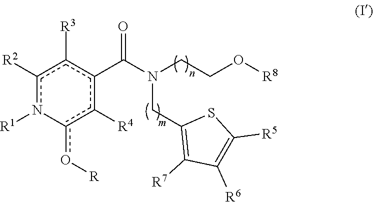 [symbol]
represents a single or a double bond, and wherein each of n, m, R, R¹, R², R³, R⁴, R⁵, R⁶, R⁷ and R⁸ is as defined in claim 38.

40. The formulation of claim 38, wherein the total amount of said at least one solvent and said at least one co-surfactant in the formulation is at least 45 wt %.

41. The formulation of claim 38, wherein the total amount of said at least one solvent and said at least one co-surfactant in the formulation is at least 50 wt %.

42. The formulation of claim 38, being devoid of water.

43. The formulation of claim 38, wherein said at least one hydrophilic surfactant is selected from the group consisting of ethoxylated fatty acids, ethoxylated castor oil and hydrogenated derivatives thereof, polysorbates, ethoxylated alkyl ethers, ethoxylated monoglycerides, polyglycerol esters and sucrose esters, and combinations thereof.

44. The formulation of claim 43, wherein the formulation comprises at least one first hydrophilic surfactant selected from the group consisting of ethoxylated castor oil and hydrogenated derivatives thereof, and at least one second hydrophilic surfactant selected from the group consisting of polysorbates and ethoxylated monoglycerides.

45. The formulation of claim 38, wherein the weight ratio between the total hydrophilic surfactants and the total co-surfactants ranges between about 3:1 and about 1:3.

46. The formulation of claim 38, wherein the at least one co-surfactant is present in the formulation in total amount ranging between about 10 wt % and about 45 wt %.

47. The formulation of claim 38, wherein said at least one co-surfactant is selected from the group consisting of polyethylene glycol 200, polyethylene glycol 400, polyethylene glycol 600, propylene glycol, phosphatidylcholine, diethyleneglycol monoethyl ether, and combinations thereof.

48. The formulation of claim 38, wherein the weight ratio between the total solvents and the total co-surfactants ranges between about 3:1 and about 1:2.

49. The formulation of claim 38, wherein said at least one solvent is selected from the group consisting of ethanol, methanol, n-propanol, benzyl alcohol, and combinations thereof.

50. The formulation of claim 38, wherein said at least one oil is present in the formulation in a concentration of no more than 4 wt %.

51. The formulation of claim 38, comprising up to 10 wt % of said compound of formula (I) or a pharmaceutically acceptable salt, isomer or tautomer thereof.

52. The formulation of claim 39, wherein n and m are 1.

53. The formulation of claim 39, wherein R², R⁷ and R⁸ are each methyl.

54. The formulation of claim 38, wherein the compound is at least one compound of formula (IA) and (IB):

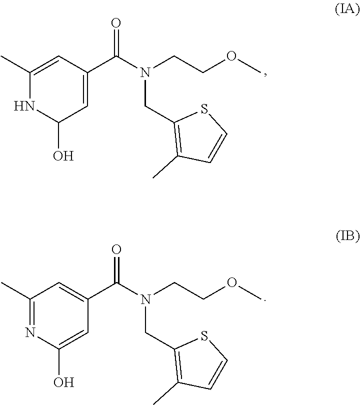

55. A preparation for oral delivery of a compound of formula (I) or a pharmaceutically acceptable salt, isomer or tautomer thereof, the preparation comprising nanodroplets of the formulation of claim 38, dispersed in a continuous phase comprising at least one aqueous diluent.

56. The preparation of claim 55, wherein said nanodroplets have an average droplet size ranging between about 5 nm and about 50 nm.

57. The preparation of claim 55, wherein said at least one aqueous diluent being selected from the group consisting of water, water for injection, saline, dextrose solution, and a buffer.
